(19) United States
(12) Patent Application Publication
(10) Pub. No.: US 2005/0048538 A1
(43) Pub. Date: Mar. 3, 2005

(54) Hypocretin receptor in regulation of sleep and treatment of sleep disorders

(76) Inventors: Emmanuel Mignot, Palo Alto, CA (US); Juliette H. Faraco, Menlo Park, CA (US); Hua Li, Union City, CA (US); Ling Lin, Mountain View, CA (US); Seiji Nishino, Palo Alto, CA (US); Hiroshi Kadatoni, Hiroshima (JP)

Correspondence Address: LAHIVE & COCKFIELD, LLP., 28 STATE STREET, BOSTON, MA, 02109, US

(21) Appl. No.: 10/868,379

(22) Filed: Jun. 14, 2004

Related U.S. Application Data
(63) Continuation of application No. 09/628,494, filed on Jul. 28, 2000, now abandoned.
(60) Provisional application No. 60/146,623, filed on Jul. 30, 1999.
(60) Provisional application No. 60/171,857, filed on Dec. 22, 1999.

(51) Int. Cl. C12Q 1/68

(52) U.S. Cl. 435/6

(57) ABSTRACT

The present invention is directed to methods for identification of compounds that affect wakefulness, attention deficit hyperactivity disorder, chronic fatigue syndrome and mood disorders (e.g., depression) through interaction with the hypocretin receptor system. The present invention is also directed to detection of abnormal levels of hypocretin in a subject, as well as detection of an abnormal immune response against hypocretin (orexins) and/or their receptors, where detection of abnormal hypocretin levels or detection of an abnormal immune response is indicative of a sleep disorder, particularly of narcolepsy. The present invention is also directed to a methods relating to the detection of a mutation or polymorphism in the gene encoding the hypocretin receptors, the detection of antibodies disrupting the function of gene encoding hypocretin receptors and hypocretin polypeptides, and the use of hypocretin biological markers in predicting treatment response using compounds interacting with the hypocretin receptor system.

### CROSS-REFERENCE TO RELATED APPLICATIONS

This application is a Continuation of U.S. patent application Ser. No. 09/628,494, filed Jul. 28, 2000, which claims the benefit of U.S. Provisional Application Ser. No. 60/146,623, filed Jul. 30, 1999, and U.S. Provisional Application Ser. No. 60/171,857, filed Dec. 22, 1999, which applications are incorporated herein by reference.

### GOVERNMENT RIGHTS

This invention was made with government support under grant nos. NS23724, NS33797, HL59601 from the National Institutes of Health. The United States Government may have certain rights in this invention.

### FIELD OF THE INVENTION

The invention relates generally to the regulation of wakefulness, sleep, narcolepsy, mood, fatigue and attention, particularly to genes products, and compounds that affect the activity of such genes and gene products in wakefulness, sleep, narcolepsy, mood, fatigue and attention.

### BACKGROUND OF THE INVENTION

Sleep and Its Disorders

Sleep is a vital behavior of unknown function that consumes one-third of any given human life. Electrophysiological studies have shown that sleep is a heterogeneous state most classically separated into rapid eye movement (REM) sleep and non-REM sleep (Dement (1994) In Principles and Practices of Sleep Medicine, Kryger, Roth and Dement, eds. (Philadelphia: W.B. Saunders Company), pp. 3-15.). REM sleep is characterized by vivid dreaming, muscle atonia, desynchronized EEG activity and REMs. Non-REM sleep is characterized by synchronized EEG activity, partial muscle relaxation and less frequent dreaming mentation (Dement, 1994, supra). The propensity to sleep or stay awake is regulated by homeostatic (sleep-debt dependent) and circadian (clock dependent) processes (Borbély, ibid, pp. 309-320.). Circadian processes are believed to be primarily generated at the genetic level within the suprachiasmatic nucleus of the hypothalamus (Klein et al. (1991). In Suprachiasmatic Nucleus The Mind's Clock (New York: Oxford University Press); Moore et al. (1998) Chronobiol. Int.15, 475-487).

While progress has been made in understanding of the generation of circadian rhythmicity, sleep generation is still poorly understood at the molecular level. The study of narcolepsy is one path to understanding sleep generation. Narcolepsy, a disabling neurological disorder affecting more than 1 in 2,000 Americans, is the only known neurological disorder that specifically affects the generation and organization of sleep. The disorder is characterized by daytime sleepiness, sleep fragmentation and symptoms of abnormal REM sleep such as cataplexy, sleep paralysis and hypnagogic hallucinations (Aldrich (1993) Prog. Neurobiol. 41, 533-541; Nishino et al. (1997) Prog. Neurobiol. 52, 27-78; Aldrich (1998) Neurology 50, S2-S7). Narcolepsy is also associated with disturbances in attention/concentration, and frequently with fatigue and depression (Roth et al. (1975) Sweitzer Archiv fir Neurologie, Neurochirugie und Psychiatrie. 116(2); 291-300; Goswami (1998). Neurology 50(suppl 1): S31 -S36). Narcolepsy also occurs in animals, and has been most intensively studied in canines (Foutz et al. (1979) Sleep 1, 413-421; Baker et al. (1985) In Brain Mechanisms of Sleep, McGinty et al. eds. (New York: Raven Press), pp 199-233; Nishino et al. supra; Cederberg et al. (1998) Vet. Rec. 142, 31-36). A large number of physiological and pharmacological studies have demonstrated a close similarity between human and canine narcolepsy. Strikingly, humans and canines with narcolepsy exhibit cataplexy, which are sudden episodes of muscle weakness (akin to REM sleep-associated atonia) that are triggered primarily by positive emotions (Foutz et al. (1979), supra; Baker et al. (1985), supra; Nishino et al. (1997) supra).

Although familial cases of narcolepsy have been reported, most human occurrences are sporadic, and conventional wisdom has suggested the disorder is multigenic and environmentally influenced (Honda et al. (1990) In Handbook of Sleep Disorder, Thorpy, ed. (New York: Marcel Dekker, Inc), pp. 217-234). One predisposing genetic factor is a specific HLA-DQ allele, HLA-DQB1*0602 (Matsuki et al. (1992) Lancet 339, 1052; Mignot et al. (1994) Sleep 17, S60-S67; Mignot et al. (1994) Sleep 17, S68-S76; Mignot et al. (1997) Sleep 20(11):1012-20). Because of the tight HLA association, the disorder in humans has been suggested to be autoimmune in nature; however all attempts to verify this hypothesis have failed (Mignot et al. (1995) Adv. Neuroimmunol. 5, 23-37). In Doberman pinschers, the disorder is transmitted as a single autosomal recessive trait with full penetrance, canarc-1 (Foutz et al. (1979), supra; Baker et al. (1985), supra).

Pharmacological, neurochemical and physiological studies implicate monoaminergic and cholinergic neurotransmissions as the main modulators in narcolepsy (Mignot (1993) J. Neurosci. 13, 1057-1064; Mignot et al. (1993) Psychopharmacology 113, 76-82; Nishino et al. (1997), supra). The human sleep disorder is currently treated symptomatically with amphetamine-like stimulants for the control of daytime sleepiness and antidepressant drugs for the control of abnormal REM sleep manifestations (e.g., cataplexy) (Aldrich, (1993), supra; Wender (1998) J Clin Psychiatry;59 Suppl 7:76-9).

Pharmacological analysis using the canine model has shown that inhibition of dopamine uptake and/or stimulation of dopamine release mediates the wake promoting effects of amphetamine-like stimulants (Nishino et al. (1997), supra), and that inhibition of noradrenergic uptake mediates the anticataplectic effects of antidepressive therapy (Mignot et al. (1993), supra). The observed effects on cataplexy parallel the well-established REM suppressant effect of adrenergic uptake inhibitors. Stimulation of cholinergic transmission using acetylcholine esterase inhibitors or direct M2 agonists also stimulates cataplexy (Nishino et al. (1997), supra). These results suggest that the pharmacological control of cataplexy, a symptom resembling REM sleep atonia, is very similar to the control of REM sleep and involves a reciprocal interaction between pontine cholinergic REM-on cells and aminergic locus coeruleus (LC) REM-off cells and their projection sites (Mignot et al. (1993), supra; Nishino et al. (1997), supra).

In order to determine the neuroanatomical basis for the sleep abnormalities observed in narcolepsy, several complementary approaches have been taken. In both human and canine subjects with narcolepsy, brain neurotransmitter levels and receptors have been measured (Miller et al. (1990) Brain Res. 509, 169-171; Aldrich (1993), supra). In narcoleptic animals, the most consistent abnormalities were observed in the amygdala where significant increases in dopamine and metabolite levels were reported in two independent studies (Miller et al., supra). These results were interpreted as suggesting decreased dopamine turnover and accumulation of dopamine in presynaptic terminals. Another important finding was the observation of increased muscarinic M2 receptors in the pontine reticular formation (Baker et al. (1985), supra; Kilduff et al. (1986) Sleep 9, 102-107), a region where cholinergic stimulation triggers REM sleep in normal animals. Local injection or perfusion of cholinergic agonists in the pontine reticular formation or the basal forebrain area triggers REM sleep and/or REM sleep atonia in narcoleptic canines (Nishino et al. (1997), supra). In narcoleptic animals, however, much lower doses can trigger muscle atonia, thus suggesting hypersensitivity to cholinergic stimulation. Furthermore, dopaminergic autoreceptor stimulation (D3) in the ventral tegmental area (VTA) induces cataplexy and sleepiness in narcoleptic but not in control canines (Reid et al. (1996) Brain Res. 733, 83-100). Because this dopaminergic system and its projection to the nucleus accumbens and other limbic structures is involved in the perception of pleasurable emotions, this observation could explain the triggering of cataplexy by positive emotions (Reid et al. (1996), supra; Nishino et al. (1997), supra). Narcolepsy may thus result from abnormal interactions between REM-on cholinergic pathways and mesocorticolimbic dopaminergic systems (Nishino et al. (1997), supra).

The Hypocretin Receptor and the Hypocretin Ligand and Feeding Patterns

As with the field of modulation of sleep patterns, the molecular basis of the regulation of energy balance and feeding patterns is beginning to be better understood. The discovery of hypocretins (orexins) and the hypocretin receptors has facilitated the unraveling of the regulatory pathways involved in eating habits. Hypocretins, which are encoded by a singe preprohypocretin mRNA transcript, are likely produced by processing of a precursor protein into two related peptides, hypocretin-1 and -2 (De Lecea et al. (1989) Proc. Natl. Acad. Sci. (USA) 95, 322-327; Sakurai et al. (1998) Cell 92, 573-585). Hypocretins are localized in the synaptic vesicle and possess neuroexcitatory effects (De Lecea et al, supra). Two orphan receptors were found to bind hypocretin-1 (also called orexin-A) and hypocretin-2 (orexin-B) with different affinity profiles (Sakurai et al., (1998), supra). The first of these receptors, now called hypocretin receptor 1 (HCRTR1), was shown to selectively bind hypocretin-1 whereas the HCRTR2 receptor binds both hypocretin-1 and 2 with a similar affinity (Sakurai et al. (1998), supra).

Initially, the finding that preprohypocretin RNA molecules and hypocretin-immunoreactive cell bodies were discretely localized to a subregion of the dorsolateral hypothalamus and a hypothesized colocalization of hypocretins with melanin concentrating hormone (MCH), a potent orexigeneic peptide, suggested a possible role of this system in the control of feeding (De Lecea et al., 1998). Furthermore, centrally administered hypocretin-1 and -2 stimulate appetite in rodents, and preprohypocretin mRNA is upregulated by fasting (Sakurai et al., 1998). However, more recent experiments suggest a more complex picture. First, the suggested initial colocalization with MCH was not substantiated by further studies (Broberger et al. (1998) J. Comp. Neurol. 402, 460-474). Second, there is controversy regarding the magnitude of the effect of hypocretins on food consumption in rodents (Lubkin et al. (1998) Biochem. Biophys. Res. Commun. 253, 241-245; Edwards et al. (1999) J. Endocrinol. 160, R7-R12; Ida (1999) Brain Res. 821, 526-529; Moriguchi et al. (1999) Neurosci. Lett. 264, 101-104; Sweet (1999) Brain Res. 821, 535-538). For example, while hypocretins stimulate short-term food intake, these peptides do not alter 24 hour total food consumption (Ida et al (1999), supra). Some authors have also suggested that hypocretins exert a shift in the diurnal pattern of food intake. The effect on energy metabolism seems to be more pronounced than that on feeding behavior (Lubkin et al. (1998), supra) and differs with the circadian time of administration (Ida et al, (1999), supra). Recent studies suggest complex interactions between hypocretins, MCH-containing neurons, neuropeptide Y, agouti gene-related protein systems and leptins in the control of feeding and energy balance (Broberger et al. (1998), supra; Beck et al. (1999) Biochem. Biophys. Res. Commun. 258, 119-122; Horvath et al. (1999). J. Neurosci. 19, 1072-1087; Kalra et al. (1999) Endocrine Rev. 20, 68-100; Marsh et al. (1999) Nature Genet. 21, 119-122; Moriguchi et al., supra; Yamamoto et al. (1999) Mol. Brain Res. 65, 14-22).

Further neuronatomical work on hypocretins and their receptors suggests a broader role than the regulation of energy balance and feeding, although the extent of that broader role had not been determined nor the specific effects that may be manifested been specifically verified. Immunocytochemical studies have shown that while the preprohypocretin-positive neurons are discretely localized in the perifornical nucleus and in the dorsal and lateral hypothalamic areas, their projections are widely distributed throughout the brain (Peyron et al. (1998) J. Neurosci. 18, 9996-10015; Date et al. (1999) Proc. Natl. Acad. Sci. (USA) 96, 748-753; Mondal et al. (1999) Biochem. Biophys. Res. Comm. 256, 495-499; Nambu et al. (1999) Brain Res. 827, 243-260; van den Pol (1999). J. Neurosci. 19, 3171-3182). Consistent with the potential role of hypocretins in the regulation of feeding, projection sites include intrahypothalamic sites such as the arcuate nucleus and paraventricular nucleus. However, other major projection sites include the cerebral cortex, the spinal cord (dorsal horn), medial nuclei groups of the thalamus, the olfactory bulb, basal forebrain structures such as the diagonal band of Brocca and the septum, limbic structures such as the amygdala and the medial part of the accumbens nucleus, and brainstem areas such as periaqueductal gray, reticular formation, pedunculopine and parabrachial nuclei, locus coeruleus, raphe nuclei, substantia nigra pars compacta and ventral tegmental area (Peyron et al., supra,; Date et al., supra; Nambu et al., supra; van den Pol, supra). A particularly dense projection is to the monoaminergic cell groups such as the raphe nucleus and the locus coeruleus (Peyron et al., supra). Of special interest is the finding that the HCRTR1 receptor transcript in rats is mostly localized in the ventromedian hypothalamic nucleus, hippocampal formation, dorsal raphe and locus coeruleus. In contrast, mRNA molecules encoding the HCRTR2 receptor are more abundant in the paraventricular nuclei and in the nucleus accumbens (Trivedi et al. (1998) FEBS Lett. 438, 71-75). Experiments using radioligand binding and immunocytochemical techniques are needed to further establish the respective pattern of expression of these receptors in relation to hypocretin projection sites.

Conclusion

Because sleep generation is poorly understood at the molecular level, the production of compounds that can be used to promote sleep or vigilance, as well as diagnosis of sleep disorders, can be difficult and imprecise. Thus, there is a need in the field for methods for identification of sleep-regulating compounds and diagnosing sleep disorders. The present invention addresses these problems in the field of sleep, as well as problems in the areas of mood and attention deficit hyperactivity disorders.

### SUMMARY OF THE INVENTION

The present invention is directed to methods for identification of compounds that affect wakefulness, attention deficit hyperactivity disorder, chronic fatigue syndrome and mood disorders (e.g., depression) through interaction with the hypocretin receptor system. The present invention is also directed to detection of abnormal levels of hypocretin in a subject, as well as detection of an abnormal immune response against hypocretin (orexins), hypocretin contiaining cells and/or hypocretin receptors, where detection of abnormal hypocretin levels or detection of an abnormal immune response is indicative of a sleep disorder, particularly of narcolepsy. The present invention is also directed to a methods relating to the detection of a mutation or polymorphism in the gene encoding the hypocretin receptors, the detection of antibodies disrupting the cells containing the hypocretin receptorsor the hypocretin polypeptides, and the use of hypocretin biological markers in predicting treatment response using compounds interacting with the hypocretin receptor system.

### BRIEF DESCRIPTION OF THE DRAWINGS

FIG. 1 is a schematic providing an overview of the region containing the canine narcolepsy gene. Human (top) and canine (bottom) chromosomal regions of conserved synteny are displayed. Human Expressed Sequence-Tag loci (ESTs) are displayed on the human map in the top panel. Key recombinant animals are listed by name in the center of the Figure. The canine narcolepsy critical region is indicated by an open box.

FIG. 2 is the map of a BAC clone contig covering the 800 kb segment known to contain canarc-1. The BAC clone sizes are drawn to scale. Selected polymorphic microsatellite markers are indicated by dotted lines. STSs for which locations were not strictly constrained are spaced at roughly equidistant intervals between constrained markers. The canine narcolepsy gene critical region is flanked by marker 26-12 (immediately distal to EST 250618) and marker 530-5 (immediately distal to EST 416643). All BAC clones were genotyped with available informative markers to determine canarc-1 associated status. Narcolepsy/control segments are indicated by solid and dashed lines, respectively. Unclassified clones are indicated by underling the clone designation.

FIG. 3 is an autoradiogram showing alternate restriction fragment length polymorphism alleles associated with the control versus narcolepsy-associated BAC clones when hybridized with an HCRTR2 probe.

FIGS. 4A, 4B and 4C are photographs showing the results of PCR amplification studies of the HCRTR2 locus in narcoleptic and control dogs. FIG. 4A: Amplification of HCRTR2 cDNA from control and narcoleptic Doberman Pinschers using primers from were designed in the 5′ and 3′ untranslated regions of the HCRTR2 gene (exon 1 and exon 7); control dog (Lane 1); narcoleptic dog (Lane 2). FIG. 4B: Amplification of narcoleptic and wild-type Doberman Pinscher genomic DNA with PCR primers flanking the SINE insertion. Lanes 1-2: wild-type Dobermans (Alex and Paris); lanes 3-4: narcoleptic Dobermans (Tasha and Cleopatra); lanes 5-6: heterozygous carrier Dobermans (Grumpy and Bob). FIG. 4C. Amplification of narcoleptic and wild-type Labrador retriever Hcrtr2 cDNAs. Lane 1: control dog; Lane 2, narcoleptic dog.

FIG. 5 is a schematic showing the deduced amino acid sequences of the hypocretin receptor 2 in wild-type dog, human, rat and narcoleptic dogs. Amino acid residues that are not identical in at least two sequences are boxed. Putative transmembrane (TM) domains are marked above the aligned sequences. Arrows indicate exon/intron boundaries in the gene structure of the dog.

FIG. 6 is a schematic showing the genomic organization of the canine Hcrtr2 locus which is encoded by 7 exons. In transcripts from narcoleptic Doberman pinschers, exon 3 is spliced directly to exon 5, omitting exon 4 (wild-type versus narc.Dob.). The genomic DNA of narcoleptic Dobermans contains an 226 bp insertion corresponding to a common canine SINE repeat element (open box) located 35 bp upstream of exon 4. The insertion of the SINE displaces a putative lariat branchpoint sequence (BPS, underlined) located at position −40 through 46 upstream of the 3′ splice site in control animals. No candidate BPS sequences are present in this vicinity in the narcolepsy-associated intron. In transcripts from narcoleptic Labrador retrievers, exon 5 is spliced directly to exon 7, omitting exon 6 (wild-type versus narc.Lab.). Genomic DNA analysis revealed a G to A transition in the 5′ splice site consensus sequence (indicated by a double underline).

FIG. 7 is a schematic providing the DNA sequence of human hypocretin polypeptide (HCRT) and indicating the polymorphism of the invention.

FIGS. 8A and 8B is a schematic providing the DNA sequence of human hypocretin receptor 1 (HCRTR1) and indicating the polymorphism of the invention.

FIGS. 9A and 9B is a schematic providing the DNA sequence of human hypocretin receptor 2 (HCRTR2) and indicating the polymorphism of the invention.

FIGS. 10A-G are photographs showing detection of Prepro-Hcrt mRNA, Melanin Concentrating Hormone (MCH) mRNA, and HLA-DR in the hypothalamus of control and narcoleptic subjects. FIGS. 10A and 10B show prepro-Hcrt mRNA in control (FIG. 10B) and narcoleptic (FIG. 10A). FIGS. 10D and 10C show MCH mRNA in the same region in control (FIG. 10D) and narcoleptic (FIG. 10C) subjects. HLA-DR staining is shown for control (FIG. 10G) and two narcoleptic (FIGS. 10 E and F) subjects. Abbreviations: f, fornix. Scale bar in (FIGS. 10A-D) represents 10 mm and in (FIGS. 10E-G) it represents 200 μm.

### DETAILED DESCRIPTION OF INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications and other forms of publically available information mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the polynucleotide” includes reference to one or more polynucleotides and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

Unless specifically indicated otherwise, “hypocretin receptor” as used herein is meant to refer to all subtypes of the hypocretin receptor, including hypocretin receptor 1 (also known as the orexin receptor 1) and the hypocretin receptor 2 (also known as the orexin receptor 2). “Hypocretin receptor” is interchangeable with “hypocretin receptor,” “hypocretin (orexin) receptor,” and with “orexin receptor.” The DNA and amino acid sequences of human hypocretin receptor 1 are provided at GenBank accession no. g4557636. The DNA and amino acid sequences of human hypocretin receptor 2 are provided at GenBank accession no. g4557638.

“Hypocretin receptor gene” as used herein is meant to encompass a nucleic acid sequence encoding a hypocretin receptor, which gene can encompass 5′ and 3′ flanking sequences and intronic sequences.

Unless specifically indicated otherwise, “hypocretin” as used herein is meant to refer to all subtypes of the naturally occurring ligands of the hypocretin receptors, including hypocretin 1 (also known as the orexin A) and hypocretin 2 (also known as the orexin B). “Hypocretin (orexin)” and “orexin” are interchangeable with “hypocretin” and with “orexin.”

As used herein the term “isolated” is meant to describe a compound of interest that is in an environment different from that in which the compound naturally occurs. □Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, preferably 75% free, and most preferably 90% free from other components with which it is naturally associated.

The term “treatment” is used herein to encompass any treatment of any disease or condition in a mammal, particularly a human, and includes: a) preventing a disease, condition, or symptom of a disease or condition from occurring in a subject which may be predisposed to the disease but has not yet been diagnosed as having it; b) inhibiting a disease, condition, or symptom of a disease or condition, e.g., arresting its development and/or delaying its onset or manifestation in the patient; and/or c) relieving a disease, condition, or symptom of a disease or condition, e.g., causing regression of the condition or disease and/or its symptoms.

By “subject” or “patient” is meant any mammalian subject for whom diagnosis or therapy is desired, particularly humans. Other subjects may include cattle, dogs, cats, guinea pigs, rabbits, rats, mice, horses, and so on. In one embodiment, subjects of particular interest are those having a sleep disorder amenable to treatment (e.g., to mitigate symptoms associated with the disorder) by, for example, administration of an agent that binds an hypocretin receptor.

By “hypocretin-related disorder,” and “disorder caused by an alteration in hypocretin receptor activity” is meant a disorder that is caused by an increase or decrease in binding of hypocretin to a hypocretin receptor relative to that found in an unaffected subject. Exemplary such disorders include, but are not necessarily limited to, sleep disorders (e.g., narcolepsy), mood disorders (e.g., depression), chronic fatigue syndrome, and hyperactivity disorders (e.g., attention deficit disorder). An increase or decrease in hypocretin receptor activity can be caused by, for example, increased or decreased levels or availability of endogenous hypocretin ligand, increased or decreased levels or availability of endogenous hypocretin receptor, alterations in a hypocretin receptor that affect the binding affinity or avidity of the receptor for hypocretin, and alterations in a hypocretin polypeptide that affect its binding affinity or avidity to a hypocretin receptor.

“LOD score” is meant to refer to an indicated probability (the logarithm of the ratio of the likelihood) that a genetic marker locus and the recited gene locus (e.g., hcrtr, particularly hcrtr2) are linked at a particular distance.

“Genetic marker” or “marker” is meant to refer to a variable nucleotide sequence (polymorphism) that is present in genomic DNA and which is identifiable with specific oligonucleotides (e.g., distinguishable by nucleic acid amplification and observance of a difference in size or sequence of nucleotides due to the polymorphism). The “locus” of a genetic marker or marker refers to its situs on the chromosome in relation to another locus as, for example, represented by LOD score and recombination fraction. Markers, as illustrated herein, can be identified by any one of several techniques know to those skilled in the art, including microsatellite or short tandem repeat (STR) amplification, analyses of restriction fragment length polymorphisms (RFLP), single nucleotide polymorphism (SNP), detection of deletion or insertion sites, and random amplified polymorphic DNA (RAPD) analysis.

“Genetic marker indicative of a mutation in the hcrtr2 gene locus” (e.g., in the context of detection of narcolepsy in canines), refers to a marker that: (a) is genetically linked and co-segregates with the hcrtr2 gene locus such that the linkage observed has a statistically significant LOD score; (b) in canines, comprises a region of canine chromosome 12, particularly between markers 26-8 and 530-3 inclusive -(c) contains a polymorphism informative for the narcoleptic genotype (e.g., comprises or is linked to a hcrtr2 mutation linked to narcolepsy); and/or (d) can be used in a linkage assay or other molecular diagnostic assays (DNA test) to identify normal alleles (wild type; (+)), and mutant (narcoleptic) alleles (by the presence of the polymorphism), and hence can distinguish narcoleptic dogs, carriers of narcoleptic alleles, and normal dogs. In that regard, markers additional to those illustrative examples disclosed herein, that map either by linkage or by physical methods so close to the hcrtr2 gene locus that any polymorphism in or with such derivative chromosomal regions, may be used in a molecular diagnostic assay for detection of hcrtr2 or carrier status.

“Co-segregate” generally means inheritance together of two specific loci; e.g., the loci are located so physically close on the same chromosome that the rate of genetic recombination between the loci is as low as 0%, as observed by statistical analysis of inheritance patterns of alleles in a mating. “Linkage” generally means co-segregation of two loci in the subject (e.g., canine breed) analyzed.

“Linkage test” and “molecular diagnostic assay” generally refer to a method for determining the presence or absence of one or more allelic variants linked with narcolpesy, e.g., with a mutant hcrtr2 gene locus, such that the method may be used for the detection of narcolepsy gene carrier status, whether through statistical probability or by actual detection of a mutated hypocretin receptor gene.

“Polymorphism” is meant to refer to a marker that is distinguishably different (e.g., in size, electrophoretic migration, nucleotide sequence, ability to specifically hybridize to an oligonucleotide under standard conditions) as compared to an analogous region from a subject of the same specieis (e.g., a dog of the same breed or pedigree).

“Nucleic acid amplification” or “amplify” is meant to refer to a process by which nucleic acid sequences are amplified in number. Several methods are known to those skilled in the art for enzymatically amplifying nucleic acid sequences including polymerase chain reaction (“PCR”), ligase chain reaction (LCR), and nucleic acid sequence-based amplification (NASBA).

“Consisting essentially of a nucleotide sequence” is meant, for the purposes of the specification or claims to refer to the nucleotide sequence disclosed, and also encompasses nucleotide sequences which are identical in sequence except for a base changes or substitutions therein while retaining the same ability to function as described, e.g., to detect a narcoleptic polymorphism, e.g., a mutant hcrtr gene linked to narcolepsy.

“Capable of hybridizing under high stringency conditions” means annealing a strand of DNA complementary to the DNA of interest under highly stringent conditions. Likewise, “capable of hybridizing under low stringency conditions” refers to annealing a strand of DNA complementary to the DNA of interest under low stringency conditions. In the present invention, hybridizing under either high or low stringency conditions generally involves hybridizing a nucleic acid sequence, with a second target nucleic acid sequence. “High stringency conditions” for the annealing process may involve, for example, high temperature and/or low salt content, which disfavor hydrogen bonding contacts among mismatched base pairs. “Low stringency conditions” generally involve lower temperature, and/or higher salt concentration than that of high stringency conditions. Such conditions allow for two DNA strands to anneal if substantial, though not near complete complementarity exists between the two strands, as is the case among DNA strands that code for the same protein but differ in sequence due to the degeneracy of the genetic code. Appropriate stringency conditions which promote DNA hybridization, for example, 6.times.SSC at about 45° C., followed by a wash of 2×SSC at 50° C. are known to those skilled in the art or can be found in Current Protocols in Molecular Biology, John Wiley & Sons, NY (1989), 6.31-6.3.6. For example, the salt concentration in the wash step can be selected from a low stringency of about 2×SSC at 50° C. to a high stringency of about 0.2×SSC at 50° C. In addition, the temperature in the wash step can be increased from low stringency at room temperature, about 22° C., to high stringency conditions, at about 65° C. Other stringency parameters are described in Maniatis, T., et al., Molecular Cloning: A Laboratory Manual, Cold Spring Harbor Laboratory Press, Cold Spring N.Y., (1982), at pp. 387-389; see also Sambrook J. et al., Molecular Cloning: A Laboratory Manual, Second Edition, Volume 2, Cold Spring Harbor Laboratory Press, Cold Spring, N.Y. at pp. 8.46-8.47 (1989).

Overview

The present invention is based on the discovery that a specific mutation in the hypocretin receptor causes narcolepsy in a canine model, that a mutation in the hypocretin peptide gene is associated with narcolepsy in humans, and that most human narcolepsy cases are associated with decreased levels of hypocretins as shown by detection of hypocretin levels (hypocretin peptide levels and preprohypocretin mRNA levels) in narcoleptic human tissues.

The findings upon which the invention is based identifies the hypocretin system as a major sleep/narcolepsy-modulating system, (e.g., hypocretin acts as sleep-modulating neurotransmitters) and opens novel potential therapeutic approaches for narcoleptic patients as well as patients suffering from other sleep disorders and/or who wish to modulate their sleep patterns (e.g., increase vigilance, facilitate sleep, etc.). These discoveries also indicate that detection of hypocretin can serve as a diagnostic tool to determine the susceptibility to a sleep disorder, to identify subject's suffering from a sleep disorder, and/or to confirm a phenotypic diagnosis of sleep disorder-susceptible or affected individuals. Because sleep, mood, fatigue and attention are tightly connected at the biochemical, clinical and therapeutic levels, the finding upon which the invention is based also indicates that the hypocretin system is involved in these related functions. Therefore, diagnostics to identify subjects susceptible to or having a sleep disorder (e.g., narcolepsy) can be applied to identify subjects susceptible to or having conditions such as mood disorders (e.g., depression), chronic fatigue syndrome, and hyperactivity disorders (e.g., attention deficit disorder). Likewise, drugs that act on hypocretins and/or hypocretin receptors to modulate hypocretin receptor activity can also serve to alleviate symptoms of mood disorders, chronic fatigue syndrome, and hyperactivity disorders. Likewise, drugs that are proposed in treatment of eating disorders (e.g., that reduce obesity) due to their interaction with hypocretin and/or hypocretin receptor(s) can be useful in the treatment of sleep disorders, as well as the above-listed exemplary related or associated disorders.

Thus the present invention is directed to, for example, the use of the hypocretin receptor in screening for compounds that bind the receptors and affect sleep patterns and wakefulness. The present invention also encompasses the detection of an abnormal or aberrant humoral or cellular immune response against hypocretins and/or their receptors, as well as detection of hypocretin levels, for the identification of subjects susceptible to a sleep disorder, particularly narcolepsy. The present invention is also directed to polymorphisms of the hypocretin receptor-encoding polynucleotide sequence for the identification of subjects susceptible to, or who are carriers for, a sleep disorder, particularly narcolepsy. The use of such polymorphisms or hypocretin measures to predict treatment responses with hypocretin receptor ligands is also encompassed by the invention. These various aspects of the invention can also find application in the diagnosis and treatment of disorders tightly associated with sleep disorders such as narcolepsy, e.g., mood disorders (e.g., depression), hyperactivity disorders (e.g., attention deficit hyperactivity disorder), and/or fatigue disorders (e.g., chronic fatigue syndrome).

Hypocretins in the Pathophysiology of Narcolepsy and the Regulation of REM Sleep

The present invention is based on the discovery that the hypocretin system (hypocretin receptors and hypocretin peptides) is involved in narcolepsy and the regulation of sleep. Prior to the discovery described herein, there was no direct evidence suggesting significant sleep/wake effects for hypocretins. The discovery that a mutation in the hypocretin receptor locus produces canine narcolepsy indicates that hypocretins and the hypocretin receptor are major neuromodulators of sleep in interaction with aminergic and cholinergic systems. This effect may be especially important during early development since, the canine model, narcolepsy typically develops between 4 weeks and 6 months of age and severity increases until animals are approximately one year old (Mignot (1993) J. Neurosci. 13, 1057-1064; Mignot et al. (1993) Psychopharmacology 113, 76-82; Riehl et al. (1998) Exp. Neurol. 152, 292-302). Furthermore, canarc-1 heterozygote animals may exhibit brief episodes of cataplexy when pharmacologically stimulated with a combination of cholinergic agonists and drugs depressing monoaminergic activity but only during early development (Mignot (1993) J. Neurosci. 13, 1057-1064; Mignot et al. (1993) Psychopharmacology 113, 76-82). Projection sites and reported hypocretin receptor localization are in agreement with a concerted effect of hypocretins, monoamines and acetylcholine on sleep-wake regulation. Central and peripheral administration of hypocretins can be potently wake-promoting and suppress REM sleep via a stimulation of a hypocretin receptor in control, but not in narcoleptic, subjects.

The Canine Narcolepsy Model and Polymorphisms in Human Narcolepsy

The phenotypes of human and canine narcolepsy and associated neurochemical abnormalities are strikingly similar (Baker 1985, supra; Nishino et al. (1997), supra). The observation than human narcolepsy is associated with low cerebrospinal fluid (CSF) hypocretin levels indicates that abnormalities in the hypocretin neurotransmission system are also involved in human cases. Mutations in the hypocretin receptor gene or other hypocretin family genes may thus be involved in some cases of human narcolepsy

The present invention also provides an example of narcolepsy-cataplexy in a human subject caused by a mutation in the signal peptide of the hypocretin polypeptide gene. This subject was non-HLA-DQB1*0602, had no CSF hypocretin levels and started narcolepsy-cataplexy at a very young age (6 months of age, as opposed to adolescence in HLA-associated narcolepsy cases). The observation that rare cases of symptomatic secondary narcolepsies are most typically associated with lesions surrounding the third ventricle (Aldrich et al. (1989) Neurology 39, 1505-1508) is also consistent with a destruction of hypocretin containing cell groups. As most cases of human narcolepsy are non-familial and strongly HLA associated (Mignot, 1997, supra) an autoimmune process directed against the hypocretin receptor or hypocretin containing cells in the hypothalamus-, or more complex neuroimmune interactions may also be involved in the pathophysiology of most cases of human narcolepsy.

Therapeutics and Methods for Identifying Therapeutics for Modulation of Sleep and/or Treatment of Narcolepsy and Other Sleep Disorders

In view of the discovery that a mutation in the hypocretin receptor and abnormal levels of hypocretin polypeptide causes narcolepsy, it follows that hypocretins, hypocretin analogues, other hypocretin receptor agonists, and hypocretin receptor antagonists offer new therapeutic avenues in narcolepsy and other sleep disorders, as well as in the modulation of sleep patterns, wakefulness, and vigilance in sleep disorder-affected and sleep-disorder unaffected individuals. Due to the association of narcolepsy with depression, chronic fatigue syndrome and attention deficit hyperactivity disorders, the discovery of the present invention also provides new therapeutic strategies for these conditions as well. A reduction of hypocretin neurotransmission can be supplemented in some cases by increasing ligand availability.

Mood Regulation Hyperactivity, Narcolepsy and Hypocretins:

An other application of the invention is in the area of mood disturbances and attention deficit hyperactivity disorder (ADHD). Narcolepsy has been previously associated with disturbances in attention/concentration and frequently fatigue and depression (Roth et al. 1975 supra; Goswami, 1998, supra). The discovery upon which the present invention is based makes it clear that mood disorders, hyperactivity disorders, and chronic fatigue syndrome can also be caused by a defect in the hypocretin system. Thus, where these disorders are so associated with a hypocretin system alteration (e.g., an alteration in levels of hypocretin peptide or hypocretin receptor production or function), such disorders can be treated and be expected to be responsive to therapy based upon alteration of the hypocretin system.

Specific aspects of the invention will now be described in more detail.

Identification of Individuals Susceptible to or Having Narcolepsy or Other Hypocretin-and/or Hypocretin Receptor-Mediated—Disorder and Identification of Subjects Having Differential Therapeutic Responses to Drugs Interacting with the Hypocretin Receptor Systems

Individuals susceptible to or having a sleep disorder caused by a hypocretin polypeptide or hypocretin receptor abnormality can be identified by (1) detection of a hypocretin receptor-encoding or hypocretin peptide sequence that contains a mutation that affects hypocretin neurotransmission function (e.g., ligand production, binding, signal transduction, and the like), (2) by detection of an abnormal immune response against hypocretin receptor, hypocretin-containing cells or its endogenous ligand (i.e. the hypocretin peptide system), and/or (3) by measuring hypocretin levels in the subject. These biological markers can also be used to predict therapeutic responsivity to drugs interacting with the hypocretin receptor system. For example, where a subject is identified as having a disorder associated with an abnormally low level of hypocretin peptide, then the subject would be expected to respond to administration of drugs that act as agonists of the hypocretin receptor or otherwise mimic or enhance the activity of hypocretin.

Diagnosis Based Upon Detection of a Polymorphism

Polymorphisms in the hypocretin receptor gene can be used to identify individuals having or susceptible to narcolepsy, and can also be used to identify carriers of the narcolepsy gene, and can similarly be used to identify a subject having a condition amenable to treatment by modulation of hypocretin receptor activity (e.g., by upregulating expression of normal hypocretin receptor, by providing an unaffected copy of the hypocretin receptor-encoding sequence, etc.). Diagnosis of such conditions or disorders can be performed by protein, DNA or RNA sequence and/or hybridization analysis of any convenient sample from a patient, e.g. biopsy material, blood sample, scrapings from cheek, etc., to examine levels of hypocretin receptor expression, and/or hypocretin receptor activity.

For example, a nucleic acid sample from a patient having a disorder that may be treated by hypocretin receptor modulation can be analyzed for the presence of a predisposing polymorphism in hypocretin receptor, e.g., a polymorphism similar to that identified in the canine model described herein. In another example, a patient may have a mutation that impairs the hypocretin peptide or its production as described below. A typical patient genotype will have at least one predisposing mutation on at least one chromosome. The presence of a polymorphic hypocretin receptor or hypocretin peptide sequence that affects the activity or expression of the gene product, and confers an increased susceptibility to an hypocretin associated disorder is considered a predisposing polymorphism. Individuals are screened by analyzing their DNA or mRNA for the presence of a predisposing polymorphism, as compared to sequence from an unaffected individual(s). Specific sequences of interest include, for example, any polymorphism that is associated with a sleep disorder, particularly narcolepsy, which polymorphisms can include, but are not necessarily limited to, insertions, substitutions and deletions in the coding region sequence, intron sequences that affect splicing, or promoter or enhancer sequences that affect the activity and expression of the protein.

A number of methods are available for analyzing nucleic acids for the presence of a specific sequence, e.g., to examine a sample for a polymorphism and/or to examine the level of hypocretin receptor mRNA production. Where large amounts of DNA are available for polymorphism analysis, genomic DNA is used directly. Alternatively, the region of interest is cloned into a suitable vector and grown in sufficient quantity for analysis.

Where expression of hypocretin or hypocretin receptors is to be analyzed, cells that express hypocretin receptor genes may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki, et al. 1985 Science 239:487; a review of current techniques may be found in Sambrook, et al. Molecular Cloning: A Laboratory Manual, CSH Press 1989, pp.14.2-14.33. Amplification may also be used to determine whether a polymorphism is present, by using a primer that is specific for the polymorphism.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. Polymorphism analysis can be performed by sequencing the nucleic acid (e.g., genomic DNA or cDNA produced from mRNA) by dideoxy or other methods, and comparing the sequence to either a neutral hypocretin receptor sequence (e.g., an hypocretin receptor/peptide sequence from an unaffected individual) or to a known, affected hypocretin receptor/peptide sequence (e.g., a hypocretin receptor sequence of a known polymorphism). Hybridization with the variant sequence may also be used to determine its presence, by Southern blots, dot blots, etc. The hybridization pattern of a control and variant sequence to an array of oligonucleotide probes immobilized on a solid support, as described in U.S. Pat. No. 5,445,934, or in WO95/35505, may also be used as a means of detecting the presence of variant sequences. Single strand conformational polymorphism (SSCP) analysis, denaturing gradient gel electrophoresis (DGGE), mismatch cleavage detection, and heteroduplex analysis in gel matrices are used to detect conformational changes created by DNA sequence variation as alterations in electrophoretic mobility. Alternatively, where a polymorphism creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels.

Analysis of relative hypocretin peptide/receptor transcriptional levels and hypocretin receptor/peptide polymorphisms can also be performed using polynucleotide arrays, and detecting the pattern of hybridization to the array, e.g., both the identity of the sequences on the array to which the sample hybridizes and/or the relative levels of hybridization (e.g., qualitative or quantitative differences in levels of expression). The hybridization pattern of a control and test sample to an array of oligonucleotide probes immobilized on a solid support, as described in U.S. Pat. No. 5,445,934, or in WO95/35505, may be used in such assays. In one embodiment of the invention, an array of oligonucleotides are provided, where discrete positions on the array are complementary to at least a portion of mRNA or genomic DNA of the hypocretin receptor/peptide loci. Such an array may comprise a series of oligonucleotides, each of which can specifically hybridize to a nucleic acid sequence, e.g., mRNA, cDNA, genomic DNA, etc. from the hypocretin receptor locus or to the hypocretin peptide locus. For example, the can comprise at least 2 different polymorphic sequences, e.g., polymorphisms located at unique positions within the locus, usually at least about 5, more usually at least about 10, and may include as many as 50 to 100 different polymorphisms. The oligonucleotide sequence on the array will usually be at least about 12 nt in length, may be the length of the provided hypocretin receptor/peptide sequences, or may extend into the flanking regions to generate fragments of 100 to 200 nt in length. For examples of arrays, see Hacia et al. 1996 Nature Genetics 14:441-447; Lockhart et al. 1996 Nature Biotechnol. 14:1675-1680; and De Risi et al. 1996 Nature Genetics 14:457-460.

The analysis of hypocretin gene polymorphisms may be used not only for diagnosing a sleep disorder but also to predict therapeutic response to hypocretin related drug treatment. For example, subjects with a given hypocretin receptor polymorphism may be shown to require much lower dose of a drug acting on hypocretin receptor to produce sleep (in case of a hypocretin receptor antagonist) or wakefulness (in case of a hypocretin receptor agonist in the treatment of narcolepsy or sleepiness, chronic fatigue syndrome, attention deficit disorder or depression) than other subjects. Analysis of hypocretin gene polymorphisms may also be indicative of the presence of other disorders tightly associated with sleep disorders in the subject, e.g., mood disorders (e.g. depression), chronic fatigue syndrome, hyperactivity disorders (e.g., attention hyperactivity deficit disorder (e.g., ADHD)), and the like.

Detection of Canine Narcolepsy Using Nucleic Acid Diagnostics

In one embodiment of the invention, comprises nucleic acid probes, nucleic acid primers, and kits comprising such probes and/or primers for detection of the canine narcolepsy/Hcrtr2 susceptibility locus. The invention is also directed to methods for identifying subjects, particularly canine subjects, susceptible to or having narcolepsy using nucleic acid diagnostic methods.

Methods

In general, the diagnostic methods of the invention are carried out by first collecting nucleic acid samples (e.g., DNA or RNA) by relatively noninvasive techniques, e.g., DNA samples can be obtained with minimal penetration into body tissues of the subjects to be tested. Common noninvasive tissue sample collection methods may be used and include withdrawing buccal cells via cheek swabs and withdrawing blood samples. Following isolation of by standard techniques, PCR is performed on the sample nucleic acid utilizing pre-designed primers that produce enzyme restriction sites on those nucleic acid samples that harbor the defective gene. Where the sample is RNA, the RNA is gerenaly first reverse transcribed to cDNA, and then PCR performed. Treatment of the amplified DNA with appropriate restriction enzymes allows one to analyze for the presence of the defective allele. One skilled in the art will appreciate that this method may be applied not only to Doberman pinschers, Laborador retrievers, and Dachsunds, but also to other breeds that may be susceptible to or carriers for narcolepsy.

Probes and Primers

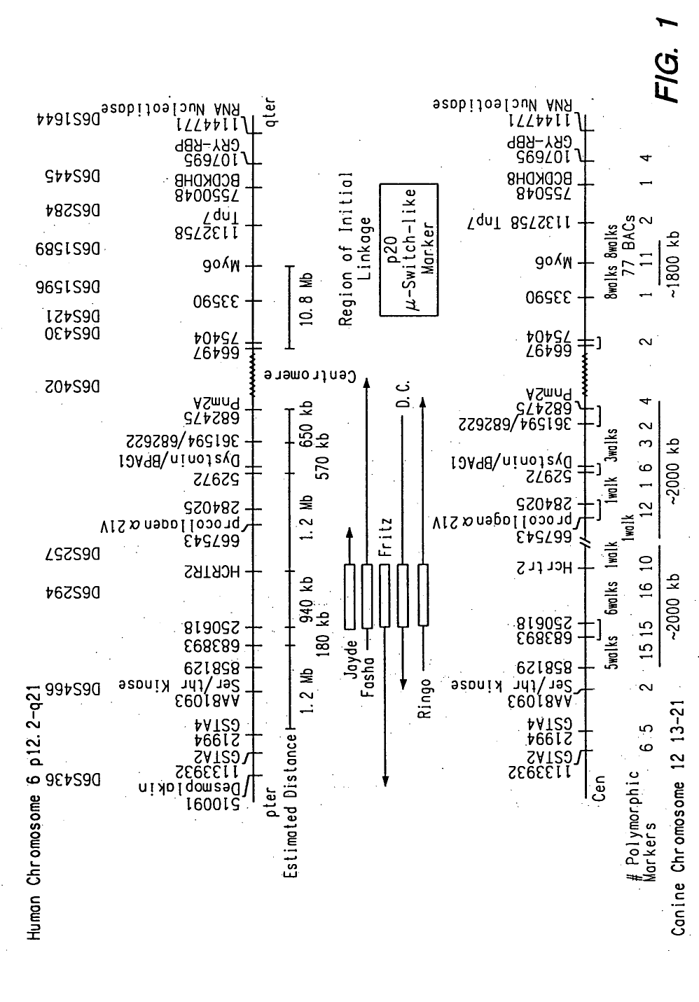

In general, the probes comprise at least a portion of a genetic marker that is linked to narcolepsy, e.g., a genetic marker indicative of a mutation in the hcrtr2 locus. The genetic markers are located on canine chromosome 12, in genomic regions that are analogous to genes or noncoding regions mapping to human chromosome 6 in the region of p12.2-q21. The region of canine chromosome 12 comprising genetic markers that are useful in the methods of the invention (“narcolepsy-informative region”) are indicated in FIG. 1, with Hcrtr2 indicating the position of the hypocretin 2 receptor gene. It will be appreciated and understood by those skilled in the art that with the identification of this region of canine chromosome 12 containing markers useful in the method of the present invention, and with the disclosure of exemplary genetic markers and the mapping of such markers to the narcolepsy-informative region (e.g., the region surrounding hcrtr2), that additional markers useful with the method of the present invention can be identified by routine linkage mapping.

Genetic markers useful in the present invention can be made using different methodologies known to those in the art. For example, using the map illustrated in FIG. 1, the narcolepsy-informative region of canine chromosome 12 (e.g., the region flanking and including the hcrtr2 gene) may be microdissected, and fragments cloned into vectors to isolate DNA segments which can be tested for linkage with the narcolepsy susceptibility locus. Alternatively, with the nucleotide sequences provided herein and described in more detail below, isolated DNA segments can be obtained from the narcolepsy-informative region of canine chromosome 12 by nucleic acid amplification (e.g., polymerase chain reaction) or by nucleotide sequencing of the relevant region of chromosome 9 (“chromosome walking”). Using the linkage test of the present invention, the DNA segments may be assessed for their ability to co-segregate with the narcolepsy susceptibility locus (e.g., a LOD score may be calculated), and thus determine the usefulness of each DNA segment in a molecular diagnostic assay for detection of narcolepsy or the carrier status.

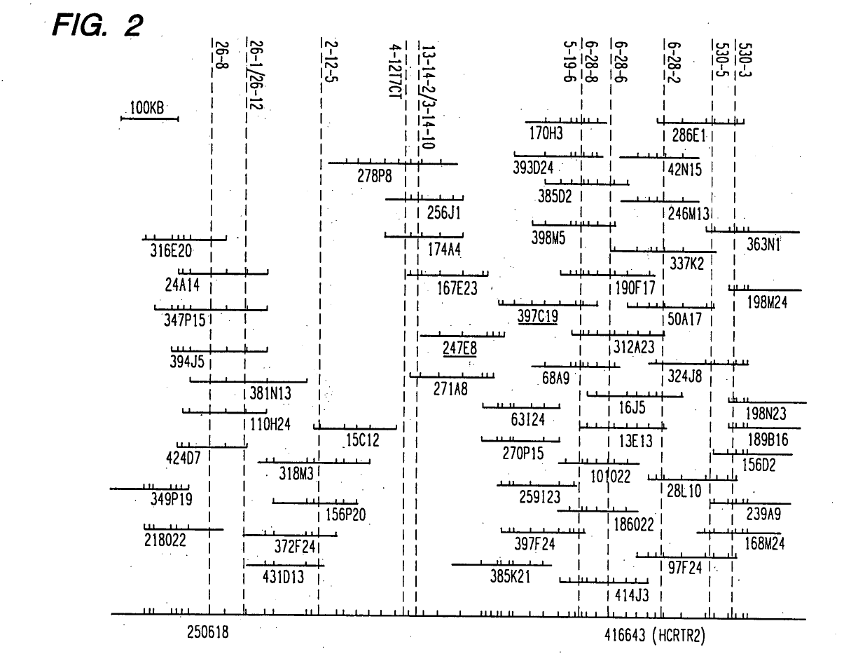

The diagnostic method of the present invention may be used to determine the genotype of an individual dog, or a set of dogs that are closely related to a dog known to be affected with narcolepsy, by identifying in each of these dogs which alleles are present using a set of marker loci linked to narcolepsy. These linked marker loci cover a region (“narcolepsy-informative region”) commencing approximately at the level of the GSTA2 gene and ending at the primase 2A gene (Pnm2A) (see FIG. 1,). Linked marker loci that are located in close proximity to the Hcrtr2 locus include microsatellite markers listed in FIG. 2 (26-8 to 530-3 inclusive).

In general, nucleic acid molecules useful as probes comprise at least about 15 contiguous nucleotides (nt), and may comprise at least about 20, 25, or 100 to 500 contiguous nucleotides. Where the probes are to be used in a hybridization assay (e.g., to provide for direct detection of a narcolepsly-linked polymorphism), the probe comprises a sequence having a unique identifier for the mutated region, e.g., the probe provides for detection of aberrant splicing or for a single or multi-nucleotide change in a canine hypocretin receptor sequence (e.g., in a hypocretin 2 receptor sequence (hcrtr2)). Preferably, the probe is capable of hybridizing under high stringency conditions to a sequence encoding a mutated canine hypocretin receptor that causes canine narcolepsy or a complement thereof.

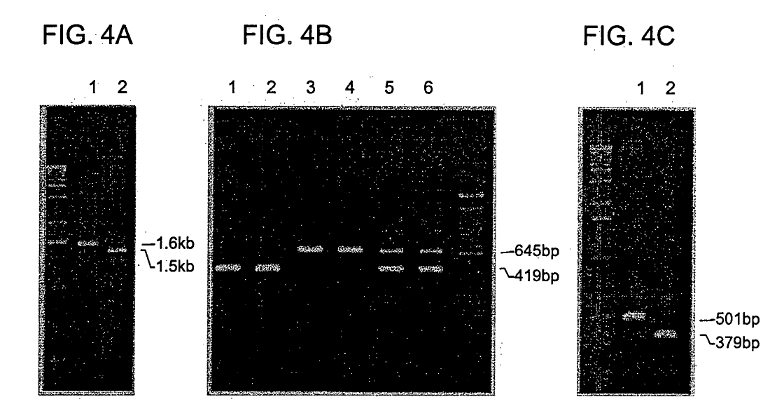

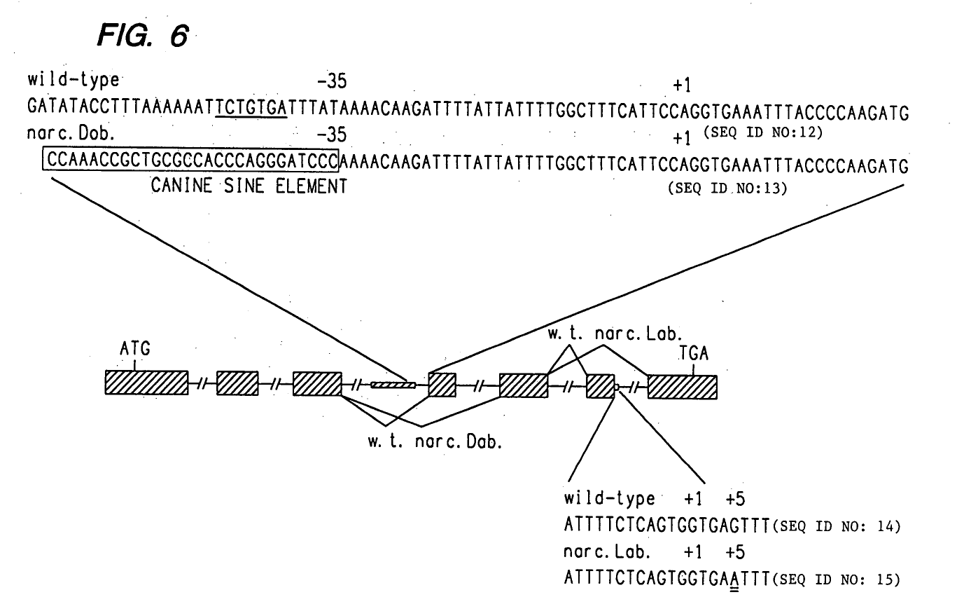

Exemplary sequences from which the probe sequence can be obtained include, but are not necessarily limited to, probes that specifically hybridize to the canine sequences listed in FIG. 6 and also included in GenBank Accession number AF164626, which provides for detection of narcolepsy in Doberman pinschers and Labradors. The Doberman narcolepsy mutation may be dectected using primers amplifying the region flanking the mutation consiting of the sine insertion described in FIG. 6 such as 554-65seqF (5′GGGAGGAACAGAAGGAGAGAATTT3′ (SEQ ID NO:3)) and R4/7-6R(110) (5′ATAGTTGTTAATGTGTACTTTAAGGC3′ (SEQ ID NO:4)) as shown in FIG. 4B. The labrador sequence(narc.Lab) listed in FIG. 6 can provide for detection a single nucleotide change within this sequence relative to wildtype (e.g., a G to A transversion at the 5′ splice site consensus sequence 3′ of exon 6,). The region containing the mutation can be amplified with primers flanking the mutated region such as

| | |
|---|---|
| 6INF(162) | (5′GACTTCATTTGGCCTTTGATTTAC3′ (SEQ ID NO:5)) |
| and | |
| 7EXR(1620) | (5′TTTTGATACGTTGTCGAAATTGCT3′ (SEQ ID NO:6)). |

Where the canine narcolepsy susceptibility locus is to be detected by amplification of the region (e.g., through RFLP analysis using PCR), exemplary primers suitable for use in the invention are provided in the table below.

Exemplary Primers Suitable for Use in Detection of Canine Narcolepsy
Susceptibility Locus

Length

| | | | |
|---|---|---|---|
| Primer | Sequence | Repeat | bp |

| | | | | |
|---|---|---|---|---|
| 530-3F1 | AAATGTCTAATCACTTTGCCCA | (SEQ ID NO:32) | (TA)25 | 150 |
| 530-3R1 | CAAATCATGTCTAATAAGGGGC | (SEQ ID NO:33) | | |
| 530-5F1 | TTGGTGGCTAGTTTTACTCTCTT | (SEQ ID NO:34) | (GAAA)320 bp | 430 |
| 530-5R1 | TGAATTCCAGTCAAATAAACAAA | (SEQ ID NO:35) | | |
| 6-28-6/F1 | TACTATTGCAGTTGGCATGCTG | (SEQ ID NO:36) | (CTTT)40 | 313 |
| 6-28-6/R1 | GCATTACTTTGATACCAAACCC | (SEQ ID NO:37) | | |
| 6-28-8/F2 | TGGACATGTCAGGGATTAAAAG | (SEQ ID NO:38) | (AT)10(ATCT)11 | 300 |
| 6-28-8/R1 | AATCCTTTGAGATTTGGAGAGG | (SEQ ID NO:39) | | |
| 6-28-2/F2 | GAATTTGTAGAGCTTGGCTAGG | (SEQ ID NO:40) | (CTTT)40 | 300 |
| 6-28-2/R2 | GATGTGTAGAGGCCATCAAGAG | (SEQ ID NO:41) | | |
| 5-19-6/F1 | CTACCAATTGTACACCCACACA | (SEQ ID NO:42) | (AT)9 . . . (GATA)15 | 227 |
| 5-19-6/R1 | TCCTTTGAGATTTGGAGAGGTA | (SEQ ID NO:43) | | |
| 4- | ctttgtgcagagtcttcttga | (SEQ ID NO:44) | (CA)5 . . . (CA)6 . . . (CA)7 | 180 |
| 12t(ca)L | | | | |
| 4- | gtggagtagctgctctaatagg | (SEQ ID NO:45) | | |
| 12t(ca)R | | | | |
| 2-12-5/F1 | CAAAGCAGCAGGGTACAAAATC | (SEQ ID NO:46) | (GAAA)100 bp | 212 |
| 2-12-5/R1 | CTTGGGATACCCCCAGTACTCC | (SEQ ID NO:47) | | |
| 26-1/F1 | GAGGCAAAATTTGCTTTTTCTC | (SEQ ID NO:48) | (CTTT)15 | 217 |
| 26-1/R1 | GCAAGTTCCAATCAACCTCAAT | (SEQ ID NO:49) | | |
| 08.26- | GCCTAACAAAATGGCACATGA | (SEQ ID NO:50) | (CAAA)7 | 182 |
| 8/T3/F | | | | |
| 08.26- | GTTGAAATTAAACTCCATCCTG | (SEQ ID NO:51) | | |
| 8/T3/R | | | | |
| 26-12/F1 | TAATCTGATTTTCCTGGAATCA | (SEQ ID NO:52) | (GAAA)180bp | 228 |
| 26-12/R1 | GGAGGCATAAATGCTAGGAAG | (SEQ ID NO:53) | | |

“Length” refers to the size of the amplified product generated using the corresponding primers.

Alternatively, where the invention involves detection of susceptibility of a canine subject to narcolepsy, the methods involve use of, and thus kits can comprise, at least one, generally at least two primers for amplification (e.g., by PCR) of a region of genomic DNA or of an mRNA (or cDNA produced from such mRNA) encoding a region of a canine hypocretin receptor gene so as to provide for detection of narcolepsy-linked mutations in the hypocretin receptor gene (e.g., the presence of a short interspersed nucleotide element (SINE) sequence, the presence of an aberrant splice junction sequence, and the like). In one embodiment, the primers are designed so that the size of the amplified gene product will be detectably different when produced from an animal having a mutant hypocretin receptor relative to a wild-type animal (i.e., an animal that does not have a hypocretin receptor mutation associated with narcolepsy. Amplification can also be accomplished using ligation amplification reaction technology (LAR) known to those skilled in the art. LAR is a method analogous to PCR for DNA amplification wherein ligases are employed for elongation in place of polymerases used for PCR.

The nucleic acid sequences described herein, particulary those useufl as hybridization probes, can be incorporated into an appropriate recombinant vector, e.g., viral vector or plasmid, which is capable of transforming an appropriate host cell, either eukaryotic (e.g., mammalian) or prokaryotic (e.g., E. coli). Such DNA may involve alternate nucleic acid forms, such as cDNA, gDNA, and DNA prepared by partial or total chemical synthesis. The DNA may also be accompanied by additional regulatory elements, such as promoters, operators and regulators, which are necessary and/or may enhance the expression of an encoded gene product. In this way, cells may be induced to over-express a hypocretin receptor or hypocretin gene, thereby generating desired amounts of a target hypocretin receptor or hypocretin protein. It is further contemplated that, for example, sequences encoding the mutated canine hypocretin receptor polypeptide sequences of the present invention may be utilized to manufacture canine mutant hypocretin receptor using standard synthetic methods.

Polypeptides in Diagnosis

One skilled in the art will appreciate that the a defective protein encoded by a defective hypocretin receptor gene of the present invention may also be of use in formulating a complementary diagnostic test for canine narcolepsy that may provide further data in establishing the presence of the defective allele. Thus, production of the defective hypocretin receptor polypeptide, either through expression in transformed host cells as described above or through chemical synthesis, is also contemplated by the present invention.

Application to Human Narcolepsy

The ordinarily skilled artisan will readily appreciate that while the above specifically describes detection of narcolepsy in dogs, the probes and primers of similar design can be used in detection of narcolepsy in humans, e.g., probes and primers for detection of truncated or otherwise mutated hypocretin receptor polypeptide-encoding sequences. In one embodiment, the probes or primers are designed to detect polymorphisms in the region between and including EST 250618 and HCRTR2 on human chromosome 6p 1 2.2-q21.

Kits for Detecting Sequence Polymorphisms

In a related aspect, the invention provides kits for detection of nucleic acid encoding a hypocretin receptor or hypocretin peptide polymorphism by hybridization of the probe to a sample suspected of comprising a nucleic acid encoding such polymorphism. Such kits can comprise, for example, a probe specific for a hypocretin receptor or hypocretin peptide polymorphism, which probe may be detectably labeled. Alternatively, a detectable label or reagent for detecting specific binding of the probe to a sample suspected of comprising a hypocretin receptor or polypeptide polymorphism can be provided as a separate component. The kit can further comprise a positive control sample, a negative control sample or both to facilitate analysis of results with the test sample. In one embodiment, the probe is bound to a solid support, and the sample suspected of containing nucleic acid comprising a hypocretin-related polymorphism (e.g., a polymorphism in a hypocretin receptor gene or a hypocretin polypeptide gene) is contacted with the support-bound probe and, after removing unbound material, formation of hybridized complexes between the probe and the test sample are detected.

The invention also provides kits for detection of a nucleic acid comprising a hypocretin receptor or hypocretin peptide polymorphism by hybridization by using a probe to amplify a nucleic acid fragment. In this embodiment, the kit can comprise primers suitable for use in amplification (e.g., using PCR) of a locus that encompasses a region of a hypocretin-related polymorphism. The primers can be detectably labeled, or the kit can further comprise an additional reagent to provide for detection of amplified product. The amplified product from the test sample is then analyzed (e.g., by determining the size or length of the amplified product) to determine if the test sample comprises a nucleic acid encoding a hypocretin-related polymorphism. For example, the size of the amplified product from the test sample is compared to a control sample (e.g., a positive control sample which comprises a hypocretin-related polymorphism, or a negative control sample which comprises a wildtype (unaffected) sample).

Diagnosis Based Upon Detection of an Abnormal Immune Response

Individuals having or susceptible to a sleep disorder mediated by hypocretin receptor system can be identified by detection of an abnormal or aberrant immune response in the subject (e.g., an autoimmune response), which may be directed against a hypocretin receptor, hypocretin-containing cells and/or an endogenous ligand of a hypocretin receptor. In one embodiment, the method of diagnosis involves the detection of autoantibodies that bind a hypocretin receptor, against a protein component expressed in hypocretin receptor containing cells or against a hypocretin receptor endogenous ligand. In a second embodiment, the method of diagnosis involves the detection of an abnormal immune cellular reactivity (for example production of cytokines in the presence of a hypocretin-related antigen) in presence of hypocretins, hypocretin system or protein component of hypocretin containing cells.

In general, such screening immunoassays are performed by obtaining a sample from a patient suspected of having an hypocretin receptor-associated disorder. “Samples,” as used herein, include tissue biopsies, biological fluids, organ or tissue culture derived fluids, and fluids extracted from physiological tissues, as well as derivatives and fractions of such fluids. Exemplary samples include, but are not necessarily limited to, cerebrospinal fluid (CSF), blood, a blood derivative, serum, plasma, and the like.

Diagnosis may be determined using a number of methods that are well known in the art. For example, antibodies against the hypocretin ligand/receptor peptides can be detected using material coated with the hypocretin ligand/receptor peptide, addition of the patient material and detection of autoantibodies using anti-human immunoglobulins. In another example, antibodies against a hypocretin receptor can be detected in a sample from a subject suspected of having or susceptible to a sleep disorder by incubating the sample with the hypocretin receptor (e.g., purified hypocretin receptor or portion thereof retaining ligand binding activity, extracts or cell lines expressing the receptor or a binding domain of a hypocretin receptor, and the like) in the presence of a detectably labeled hypocretin receptor ligand (e.g., detectably labeled hypocretin (orexin)). The presence of antibody-antigen complex is then detected with a secondary antibody (anti-human immunoglobulin antibody) against the receptor, and/or the ability of the sample to compete for hypocretin receptor binding with the detectably labeled hypocretin receptor ligand (or inhibit such binding) is assessed. This can be accomplished using any of a variety of methods known in the art (e.g., fluorescence activated cell sorter (FACS), ELISA, etc.). The presence of anti-hypocretin receptor antibodies or anti-hypocretin antibodies in the sample is indicative of a sleep disorder, or susceptibility to a sleep disorder, in the subject.

Kits for Detecting Aberrant Immune Responses that Affect Hypocretin System Function

In a related aspect, the invention provides kits for detection of an aberrant immune response (e.g., an autoimmune response) that affects hypocretin-related activity in a subject. Such kits can comprise, for example, a specific binding reagent (e.g., a prehypocretin protein, a hypocretin peptide or antigenic fragment thereof, a hypocretin receptor or antigenic fragment thereof, or any antigenic protein component contained in hypocretin containing cell) for detecting the presence of anti-hypocretin system antibodies in a sample obtained from the subject. The specific binding reagent may be detectably labeled or a detectable label for detection of binding reagent specifically bound to a hypocretin-related component of the sample. The kit can further comprise a positive control sample, a negative control sample or both to facilitate analysis of results with the test sample. In one embodiment, the specific binding reagent is bound to a solid support, and the sample suspected of containing an anti-hypocretin system antibody is contacted with the support-bound probe. After removing unbound material, formation of hybridized complexes between the probe and the test sample are detected.

Diagnosis Based Upon Detection of Hypocretin Levels

The subjects having or susceptible to a sleep disorder (e.g., narcolepsy) can be identified by assessing levels of hypocretin in a subject. In general, the assays contemplated by the invention involve contacting a test sample from a subject suspected of having or being susceptible to a sleep disorder such as narcolepsy with a hypocretin binding-molecule (most typically antibodies), and detecting complexes (e.g., by radioimmunoassays). Other assays covered by the invention may indirectly measure hypocretin levels by measuring the biological activity of the peptide using in vivo biological tests (e.g. using tissue known to express a specific and measurable response to hypocretin stimulation via hypocrerin receptors) or by measuring the expression of such peptide or receptor in a biological sample. The assay can involve detection of preprohypocretin and all its derivatives (e.g. hypocretin-1, hypocretin-2, both hypocretin-1 and hypocretin-2 and other peptide fragments derived from preprohypocretin). As used in the context of the detection assay, “hypocretin” is meant to encompass detection of either one or both forms of hypocretin or any preprohypocretin derivatives. The assay can also involve detection of hypocretin-producing and/or hypocretin-containing cells in patient tissue (e.g., using imaging technology such as Magnetic Resonance Imaging, Positron Emission Tomography and the like) to, assess distraction of such cells and/or measuring levels of hypocretin receptor or hypocretin peptide expression using such imaging methods or other suitable methods known in the art.

Detection of a level of hypocretin that is decreased or increased relative to a level in a normal subject is indicative of a sleep disorder, particularly narcolepsy, in the subject. For example, detection of decreased, especially dramatically decreased hypocretin levels in a subject is indicative of narcolepsy. The biological marker may also be used to predict treatment response to hypocretin receptor drugs. For example, a narcoleptic subject with no detectable hypocretin levels in his cerebrospinal fluid may have a better therapeutic response to hypocretin receptor agonists that a subject with normal hypocretin level. While direct detection of hypocretin is described herein, it is to be understood that detection of other polypeptides or other molecules that provide for indirect assessment of hypocretin levels is also contemplated by the invention. For example, detection of a polypeptide (other than mature hypocretin) that results from processing of preprohypocretin can serve as a surrogate marker for hypocretin levels.

Any sample that is suitable for detection of hypocretin levels either qualitatively or quantitatively is suitable for use in the method of the invention. Exemplary samples suitable for use in the detection assay of the invention include, but are not necessarily limited to cerebrospinal fluid (CSF), blood, seminal fluid, urine, white blood cells and the like. The patient sample may be used directly, or diluted as appropriate, e.g., about 1:10 and usually not more than about 1:10,000. Immunoassays may be performed in any physiological buffer, e.g. PBS, normal saline, HBSS, PBS, etc.

Methods for detection of hypocretin involve the detection of binding between hypocretin and a hypocretin-specific binding molecule (e.g., anti-hypocretin antibodies or fragments thereof that retain antigen binding specificity, hypocretin receptors or fragments thereof that retains hypocretin binding specificity, and the like) or other methods. Detection of a level of hypocretin that is lower or higher relative to a normal hypocretin level (e.g., a hypocretin level in a non-affected subject) is indicative of a sleep disorder, particularly narcolepsy, in the subject. As will be readily apparent to the ordinarily skilled artisan upon reading the present specification, detection of hypocretin can be accomplished in a variety of ways.

In one embodiment, a conventional sandwich type assay is used. A sandwich assay is performed by first immobilizing proteins from the test sample on an insoluble surface or support. The test sample may be bound to the surface by any convenient means, depending upon the nature of the surface, either directly or indirectly. The particular manner of binding is not crucial so long as it is compatible with the reagents and overall methods of the invention. They may be bound to the plates covalently or non-covalently, preferably non-covalently.

The insoluble supports may be any compositions to which the test sample polypeptides can be bound, which is readily separated from soluble material, and which is otherwise compatible with the overall method of detecting and/or measuring hypocretin. The surface of such supports may be solid or porous and of any convenient shape. Examples of suitable insoluble supports to which the receptor is bound include beads, e.g., magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

After adding the patient sample or fractions thereof to the support, non-specific binding sites on the insoluble support, i.e. those not occupied by sample polypeptide, are generally blocked. Preferred blocking agents include non-interfering proteins such as bovine serum albumin, casein, gelatin, and the like. Alternatively, several detergents at non-interfering concentrations, such as Tween, NP40, TX100, and the like may be used.

Samples, fractions or aliquots thereof can be added to separately assayable supports (for example, separate wells of a microtiter plate). Preferably, a series of standards, containing known concentrations of hypocretin is assayed in parallel with the samples or aliquots thereof to serve as controls and to provide a means for quantitating the amounts of hypocretin in the test sample. Generally from about 0.001 ml to 1 ml of sample, diluted or otherwise, is sufficient, usually about 2 ml to 50 ml sufficing. Preferably, each sample and standard will be added to multiple wells so that mean values can be obtained for each.

After the test sample polypeptides are immobilized on the solid support, a hypocretin-specific binding molecule that specifically binds hypocretin (e.g., an anti-hypocretin specific antibody (e.g., an anti-hypocretin-1 monoclonal or polyclonal antibody, preferably a monoclonal antibody) or other hypocretin-binding molecule (e.g. a hypocretin receptor or fragment thereof)) is added. For sake of clarity in this example, the hypocretin-specific binding molecule is a monoclonal antibody that specifically binds hypocretin. However, it is to be understood that other hypocretin-specific binding molecules can be readily substituted for the antibody in this example. Methods for generating antibodies that specifically bind hypocretin are well known in the art, and need not be described in detail here. Furthermore, anti-hypocretin antibodies are commercially available and can be used in the methods of the present invention.

The incubation time of the sample and the anti-hypocretin first receptor should be for at time sufficient for binding to the insoluble polypeptide to form an antibody-hypocretin complex. Generally, from about 0.1 to 3 hr is sufficient, usually 1 hr sufficing.

After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, formation of anti-hypocretin antibody/hypocretin complexes to the sample can be detected by virtue of a detectable label on the anti-hypocretin antibody. Where the anti-hypocretin antibody is not detectably labeled, antibody binding can be detected by contacting the sample with a solution containing first receptor-specific second receptor (e.g., anti-hypocretin antibody-specific second receptor), in most cases a secondary antibody (i.e., an anti-antibody). The second receptor may be any compound which binds antibodies with sufficient specificity such that the bound antibody is distinguished from other components present. In one embodiment, second receptors are antibodies specific for the anti-hypocretin antibody, and may be either monoclonal or polyclonal sera, e.g. goat anti-mouse antibody, rabbit anti-mouse antibody, etc.

The antibody-specific second receptors are preferably labeled to facilitate direct, or indirect quantification of binding. Examples of labels which permit direct measurement of second receptor binding include light-detectable labels, radiolabels (such as ³H or ¹²⁵I), fluorescers, dyes, beads, chemiluminescers, colloidal particles, and the like. Examples of labels which permit indirect measurement of binding include enzymes where the substrate may provide for a colored or fluorescent product. In one embodiment, the second receptors are antibodies labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

Alternatively, the second receptor may be unlabeled. In this case, a labeled second receptor-specific compound is employed which binds to the bound second receptor. Such a second receptor-specific compound can be labeled in any of the above manners. It is possible to select such compounds such that multiple compounds bind each molecule of bound second receptor. Examples of second receptor/second receptor-specific molecule pairs include antibody/anti-antibody and avidin (or streptavidin)/biotin. Since the resultant signal is thus amplified, this technique may be advantageous where only a small amount of hypocretin is present, or where the background measurement (e.g., non-specific binding) is unacceptably high. An example is the use of a labeled antibody specific to the second receptor. More specifically, where the second receptor is a rabbit anti-allotypic antibody, an antibody directed against the constant region of rabbit antibodies provides a suitable second receptor specific molecule. The anti-Ig will usually come from any source other than human, such as ovine, rodentia, particularly mouse, or bovine.

The volume, composition and concentration of anti-antibody solution provides for measurable binding to the antibody already bound to receptor. The concentration will generally be sufficient to saturate all antibody potentially bound to hypocretin. When antibody ligands are used, the concentration generally will be about 0.1 to 50 mg/ml, preferably about 1 mg/ml. The solution containing the second receptor is generally buffered in the range of about pH 6.5-9.5. The solution may also contain an innocuous protein as previously described. The incubation time should be sufficient for the labeled ligand to bind available molecules. Generally, from about 0.1 to 3 hr is sufficient, usually 1 hr sufficing.

After the second receptor or second receptor-conjugate has bound, the insoluble support is generally again washed free of non-specifically bound second receptor, essentially as described for prior washes. After non-specifically bound material has been cleared, the signal produced by the bound conjugate is detected by conventional means. Where an enzyme conjugate is used, an appropriate enzyme substrate is provided so a detectable product is formed. More specifically, where a peroxidase is the selected enzyme conjugate, a preferred substrate combination is H₂O₂ and is O-phenylenediamine which yields a colored product under appropriate reaction conditions. Appropriate substrates for other enzyme conjugates such as those disclosed above are known to those skilled in the art. Suitable reaction conditions as well as means for detecting the various useful conjugates or their products are also known to those skilled in the art. For the product of the substrate O-phenylenediamine for example, light absorbance at 490-495 nm is conveniently measured with a spectrophotometer.

The absence or presence of antibody binding may be determined by various methods that are compatible with the detectable label used, e.g., microscopy, radiography, scintillation counting, etc. Generally the amount of bound anti-hypocretin antibody detected will be compared to control samples (e.g., positive controls containing known amounts of hypocretin or negative controls lacking such polypeptides). The presence of decreased levels of bound anti-hypocretin antibody indicative of decreased levels of hypocretin in the sample, which in turn is indicative of a sleep disorder, particularly narcolepsy in the subject from whom the sample was obtained. Usually at least about a 2-fold decrease, often about a 4- to 5-fold decrease, generally a decrease in hypocretin levels to an undetectable level (e.g., less than about 40 pg/ml) in the test sample relative to hypocretin levels associated with normal subjects (e.g., subjects not affected by a sleep disorder such as narcolepsy) is indicative of a sleep disorder, particularly narcolepsy in a subject. In general, a 2-5 fold increase is also indicative of narcolepsy. The severity of the sleep disorder or the treatment response may also be directly correlated with the level of hypocretin in the sample.

Variations of the hypocretin detection assay of the invention as described above will be readily apparent to the ordinarily skilled artisan. For example, a competitive assay may be used, e.g., radioimmunoassay, etc. In addition to the patient sample, a competitor to hypocretin for binding to the hypocretin-specific binding molecule is added to the reaction mix. Usually, the competitor molecule will be labeled and detected as previously described, where the amount of competitor binding will be proportional to the amount of hypocretin in the sample. In one embodiment, the competitor molecule is a detectably labeled hypocretin polypeptide or fragment thereof that specifically binds the selected hypocretin-specific binding molecule to be used in the assay. Suitable detectable labels include those described above (e.g., radioactive labels, fluorescent labels, and the like). The concentration of competitor molecule will be from about 10 times the maximum anticipated hypocretin concentration to about equal concentration in order to make the most sensitive and linear range of detection.

Another alternative protocol is to provide hypocretin-specific binding molecules bound to the insoluble surface. After immobilization of the hypocretin-specific binding molecule on the insoluble support, the test sample is added, the sample incubated to allow binding of hypocretin, and complexes of hypocretin-hypocretin-specific binding molecule detected as described above.

In yet another alternative embodiment, the detection assay may be carried out in solution. For example, anti-hypocretin antibody is combined with the test sample, and immune complexes of antibody and hypocretin are detected. Other immunoassays (e.g., Ouchterlony plates or Western blots may be performed on protein gels or protein spots on filters) are known in the art and may find use as diagnostics.

In a related embodiment, the invention provides kits for detecting hypocretin in a sample obtained from a subject, where the kit can comprise as its components any or all of the reagents described above. In some embodiments, the reagents may be bound to a soluble support where appropriate, and may be detectably labeled or provided in conjunction with an additional reagent to facilitate detection.

Identification of Compounds that Bind the Orexin Receptor and Regulate Wakefulness

In another aspect the invention features a method for identification and use of wakefulness-promoting (hypocretin receptor agonist) and sleep-promoting (hypocretin receptor antagonists) agents by screening candidate agents for the ability to bind the hypocretin receptor in vitro and/or in vivo. Based on the observation that narcolepsy is associated with depression, fatigue and attention defect, and that hypocretins interact with monoaminergic systems involved in the regulation of these functions, the invention also features a method for identification and use of hypocretin receptor agonists in the treatment of attention deficit hyperactivity disorder, chronic fatigue syndrome and depression. Exemplary screening assays are described in more detail below.

Drug Screening

The animal models described herein, as well as methods using the hypocretin receptor in vitro, can be used to identify candidate agents that affect hypocretin receptor expression (e.g., by affecting hypocretin receptor promoter function) or that otherwise affect hypocretin receptor activity, e.g., by binding to stimulate or antagonize hypocretin receptor activity (e.g., the binding agent acts as an hypocretin receptor agonist and thus promotes wakefulness, or the binding agent acts as an hypocretin receptor antagonist and promotes sleep). Agents of interest include those that enhance, inhibit, regulate, or otherwise affect hypocretin receptor activity and/or expression. Agents that alter hypocretin receptor activity and/or expression can be used to, for example, treat or study disorders associated with decreased hypocretin receptor activity. “Candidate agents” is meant to include synthetic molecules (e.g., small molecule drugs, peptides, or other synthetically produced molecules or compounds, as well as recombinantly produced gene products) as well as naturally-occurring compounds (e.g., polypeptides, endogenous factors present in mammalian cells, hormones, plant extracts, and the like) and derivatives of such naturally-occurring compounds (e.g., hypocretin derivatives or analogues having altered receptor binding characteristics, etc)

Agents that stimulate or otherwise increase hypocretin receptor activity (e.g., hypocretin receptor “agonists,” which includes, but are not necessarily limited to, agents that bind to and stimulate hypocretin receptor, agents that promote binding of endogenous hypocretin ligand, agents that increase hypocretin receptor expression, and the like) are of interest as agents that enhance wakefulness. Agents that inhibit hypocretin receptor activity (e.g., hypocretin receptor “antagonists,” which includes, but are not necessarily limited to, agents that bind to hypocretin receptor but do not substantially stimulate the activity of the receptor, agents that block binding of hypocretin receptor agonists, agents that decrease hypocretin receptor expression, and the like) are of interest as agents that promote sleep. Agonistic and antagonistic agents can be used for the treatment of sleep disorders and/or for administration to subjects who wish to enhance their vigilance or promote sleep, but who are not affected or fully affected by a sleep disorder.

Exemplary embodiments of the drug screening assays of the invention will now be described in more detail.

Drug Screening Assays

Of particular interest in the present invention is the identification of agents that have activity in affecting hypocretin receptor expression and/or function. Drug screening can be designed to identify agents that provide a replacement or enhancement for hypocretin receptor function, or that reverse or inhibit hypocretin receptor function. Of particular interest are screening assays for agents that have a low toxicity for human cells.

The term “agent” as used herein describes any molecule with the capability of altering or mimicking the expression or physiological function of hypocretin receptor. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, including, but not limited to, organic molecules (e.g., small organic compounds having a molecular weight of more than 50 and less than about 2,500 daltons), peptides, antisense polynucleotides, and ribozymes, and the like. Candidate agents can comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: polynucleotides, peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Screening of Candidate Agents In Vitro

A wide variety of in vitro assays may be used to screen candidate agents for the desired biological activity, including, but not limited to, in vitro binding assays using labeled ligands, measurements of intracellular effects in cells expressing or having surface hypocretin receptors (e.g., calcium imaging, GTP binding, second messenger systems, etc.), protein-DNA binding assays (e.g., to identify agents that affect hypocretin receptor expression), electrophoretic mobility shift assays, immunoassays for protein binding, and the like. For example, by providing for the production of large amounts of hypocretin receptor protein, one can identify ligands or substrates that bind to, modulate or mimic the action of the proteins. The purified protein may also be used for determination of three-dimensional crystal structure, which can be used for modeling intermolecular interactions, transcriptional regulation, etc.

The screening assay can be a binding assay, wherein one or more of the molecules may be joined to a label, and the label directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assays described herein. Where the assay is a binding assay, these include reagents like salts, neutral proteins, e.g. albumin, detergents, etc that are used to facilitate optimal protein-protein binding, protein-DNA binding, and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Many mammalian genes have homologs in yeast and lower animals. The study of such homologs“physiological role and interactions with other proteins in vivo or in vitro can facilitate understanding of biological function. In addition to model systems based on genetic complementation, yeast has been shown to be a powerful tool for studying protein-protein interactions through the two hybrid system described in Chien et al. 1991 Proc. Natl. Acad. Sci. USA 88:9578-9582. Two-hybrid system analysis is of particular interest for exploring transcriptional activation by hypocretin receptor proteins and to identify cDNAs encoding polypeptides that interact with hypocretin receptor.

In one embodiment, the screening assay is a competitive binding assay to identify agents that compete with hypocretin for binding of the hypocretin receptor.

Screening of Candidate Agents In Vivo

Candidate agents can be screened in an animal model of a sleep disorder (e.g., in the narcoleptic canine model described in the Examples below; in animals that are transgenic for an alteration in hypocretin receptor, e.g., a transgenic hypocretin receptor “knock-out,” hypocretin receptor “knock-in,” hypocretin receptor comprising an operably linked reporter gene, and the like).

In one embodiment, screening of candidate agents is performed in vivo in a transgenic animal described herein. Transgenic animals suitable for use in screening assays include any transgenic animal having an alteration in hypocretin receptor expression, and can include transgenic animals having, for example, an exogenous and stably transmitted human hypocretin receptor gene sequence, a reporter gene composed of a (removed human) hypocretin receptor promoter sequence operably linked to a reporter gene (e.g., β-galactosidase, CAT, or other gene that can be easily assayed for expression), or a homozygous or heterozygous knockout of an hypocretin receptor gene. The transgenic animals can be either homozygous or heterozygous, preferably homozygous, for the genetic alteration and, where a sequence is introduced into the animal's genome for expression, may contain multiple copies of the introduced sequence. Where the in vivo screening assay is to identify agents that affect the activity of the hypocretin receptor promoter, the hypocretin receptor promoter can be operably linked to a reporter gene (e.g., luciferase) and integrated into the non-human host animal's genome or integrated into the genome of a cultured mammalian cell line.

In general, the candidate agent is administered to the animal, and the effects of the candidate agent determined. The candidate agent can be administered in any manner desired and/or appropriate for delivery of the agent in order to effect a desired result. For example, the candidate agent can be administered by injection (e.g., by injection intravenously, intramuscularly, subcutaneously, or directly into the tissue in which the desired affect is to be achieved), orally, or by any other desirable means. Normally, the in vivo screen will involve a number of animals receiving varying amounts and concentrations of the candidate agent (from no agent to an amount of agent hat approaches an upper limit of the amount that can be delivered successfully to the animal), and may include delivery of the agent in different formulation. The agents can be administered singly or can be combined in combinations of two or more, especially where administration of a combination of agents may result in a synergistic effect.

The effect of agent administration upon the transgenic animal can be monitored by assessing hypocretin receptor function as appropriate (e.g., by examining expression of a reporter or fusion gene), or by assessing a phenotype associated with the hypocretin receptor expression (e.g., wakefulness, vigilance, sleep patterns, etc.). Methods for assaying levels of a selected polypeptide, levels of enzymatic activity, and the like are well known in the art.

Where the in vivo screening assay is to identify agents that affect the activity of the hypocretin receptor promoter and the non-human transgenic animal (or cultured mammalian cell line) comprises an hypocretin receptor promoter operably linked to a reporter gene, the effects of candidate agents upon hypocretin receptor promoter activity can be screened by, for example, monitoring the expression from the hypocretin receptor promoter (through detection of the reporter gene). Alternatively or in addition, hypocretin receptor promoter activity can be assessed by detection (qualitative or quantitative) of hypocretin receptor mRNA or protein levels.

Identified Candidate Agents

Compounds having the desired pharmacological activity may be administered in a physiologically acceptable carrier to a host for treatment of a condition that is amenable to treatment by modulation of hypocretin receptor activity (e.g., stimulation of hypocretin receptor activity or inhibition of hypocretin receptor activity). The compounds may also be used to enhance hypocretin receptor function.

Examples of conditions that can be treated using the therapeutic agents described herein include, but are not necessarily limited to, sleep disorders (e.g., narcolepsy, hypersomnia, insomnia, obstructive sleep apnea syndrome, and the like), depression, chronic fatigue syndrome, attention deficit hyperactivity disorder as well as conditions of subjects that would not necessarily be diagnosed as having a classical sleep disorder, but who desire to alter their sleep patterns (e.g., to promote sleep, to promote wakefulness, to promote vigilance, etc.).

The therapeutic agents may be administered in a variety of ways, orally, topically, parentally e.g. subcutaneously, intraperitoneally, by viral infection, intravascularly, etc. Oral and inhaled treatments are of particular interest. Depending upon the manner of introduction, the compounds may be formulated in a variety of ways. The concentration of therapeutically active compound in the formulation may vary from about 0.1-100 wt. %. The therapeutic agents can be administered in a single dose, or as multiple doses over a course of treatment.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Identification and Detection of Canine Narcolepsy Mutations Altering the Hypocretin Receptor 2

a) Methods and Materials

The following Methods and Materials were used in the course of performing the work described in Example 1.

i) Canine Subjects and Genetic Linkage Analysis:

Backcross narcoleptic Dobermans and Labradors were produced in our breeding colony at the Center for Narcolepsy as described in Cederberg et al., (1998), supra. The procedure to determine phenotypic status for these dogs is described in Mignot (1993) J. Neurosci. 13, 1057-1064; Mignot et al. (1993) Psychopharmacology 113, 76-82. All experimental procedures were done in accordance with the NIH guidelines for laboratory animal care. Two familial cases of canine narcolepsy were reported to our attention for therapeutic advice by a veterinarian and a breeder respectively (see text). Diagnosis for these cases was verified by phone interview and breeding into the colony whenever possible (one of two cases). Linkage analysis was performed as described in Mignot (1991) Proc. Natl. Acad. Sci. (USA) 88, 3475-3478, with kind assistance of Neil Risch (Stanford, Calif.).

ii) Radiation Hybrid Mapping of EST Candidate Loci and Human EST Clone Selection:

At the time our project started, EST mapping information obtained from various online sources (e.g., Genemap '96; Whitehead Institute; Sanger Center) was often contradictory or of a low resolution, so that the precise location of these genes was not reliably known. Radiation hybrid (RH) mapping is a simple and reliable method for mapping genes (Cox et al. (1990) Science 250, 245-250) and whole genome RH panels have been developed to quickly localize genes to their unique chromosomal location. We made use of the 83 hybrid Stanford G3 radiation hybrid panel to map a number of candidate anchor ESTs in order to verify that the EST lay within a relatively large region of interest (GSTA4-PRIM2A) and to attempt to characterize the relative order of our selected EST loci. One μl of each hybrid DNA, plus positive and negative control parental cell DNA, was transferred for PCR in a 96 well PCR plate. PCR primers published in dbSTS (on the National Center for Biotechnology Information website) the previously mentioned online sites, were obtained and used in a 10 μl PCR reaction to amplify these genes. Amplification was performed at: 95° C. 5 min; 35 cycles of 95° C. 30 sec, 52-62° C. (primer dependent) 30 sec, 72° C. 30 extension of 72° C. for 10 minutes. The reaction products were run on a 2% agarose gel and scored for the presence of a human specific PCR product of the expected size. A positive result was denoted with the number one (1), and negative result a number two (2), and an ambiguous result was given an R. The data vector was submitted to the Stanford Human Genome Center (SHGC) radiation hybrid server in order to perform a two-point analysis with the genetic markers contained in their database. The most tightly linked RH marker and the estimated distance from that marker were returned by the RH server.

iii) Screening of the Human BAC Library with Human EST Probes:

Human IMAGE consortium clones mapping to the pericentromeric region of human chromosome 6 were identified through scrutiny of available data on the internet from maps constructed by the Whitehead Institute for Genomic Research, GeneMap 96, GeneMap 98 the Sanger Centre, the Stanford Human Genome Center and through Unigene. Selected clones were obtained from Research Genetics (Huntsville, Ala.) and verified through sequence analysis of extracted DNA. IMAGE clone inserts were excised and band purified on agarose gels (Qiaquick spin columns, Qiagen) for use as hybridization probes. Probes were evaluated by hybridization of strips of Southern blotted canine genomic DNA. Those not producing high background signal or obvious nonspecific hybridization signals were used to screen the Canine Genomic BAC library. Hybridizations and washes were performed in standard BAC library buffers as described in Li et al. (1999) Genomics 58, 9-17, but were carried out at 51-53° C. to reduce stringency. Positive clones were selected from the library, streaked onto LB plates supplemented with chloramphenicol and DNA was extracted from 5 ml minicultures of single clones. BAC DNA was digested with EcoRI and SacI, electrophoresed in agarose gels and Southern blotted onto nylon filters. Filters were hybridized with the appropriate EST probes to identify true positive clones. Positive clones were grouped into bins based on patterns produced by ethidium bromide staining and hybridization results. Clones from each bin were further characterized through two color chromosomal Fluorescence In Situ Hybridization using a previously characterized CFA12 BAC (as described in Li et al., 1999, supra) clone as a positive control to verify that the clones were in the narcolepsy region. In most cases, plasmid minilibrary clones were also hybridized with the EST probes and resulting subclones were sequenced in order to identify homologous canine exon sequences.

iv) Canine Fluorescence In Situ Hybridization:

BAC clones were analyzed by FISH on canine metaphase spreads to confirm location onto CFA12. Briefly, BAC clones were labeled with digoxygenin or biotin conjugated nucleotides using nick translation kits (Boehringer Mannheim and Gibco BRL). Following nick translation, 100-500 ng of labeled DNA was twice precipitated together with 10 μg of sheared total dog genomic DNA and 1 μg salmon sperm DNA. After resuspension with 10 μl formamide hybridization buffer, DNAs were denatured for 10 minutes at 70° C., directly transferred to 37° C. and allowed to pre-anneal for at least 15 minutes. Canine metaphase chromosome spreads were prepared from peripheral lymphocytes according to standard methods (see, e.g., Barch (1997). In AGT Cytogenetics Laboratory Manual (New York: Lippincott-Raven). Prior to hybridization, chromosome slides were treated with RNase and subjected to dehydration in an ethanol series (70, 80, 90, 100%) for 5 minutes in each concentration, and allowed to air dry. The chromosome spreads were next denatured in 70% formamide, 2×SSC at 65° C. for 5 minutes, quenched in iced 70% ethanol and again dehydrated in an ethanol series. After air drying, slides were hybridized to labeled BACs at 37° C. overnight. Some BAC clones were analyzed by sequential G-banding-FISH to allow specific chromosomal assignments. GTW-banded slides were photographed and de-stained by 3 one-minute washes in 3:1 methanol/acetic acid. Slides were then dried and treated in 2×SSC at 37° C. for 30 minutes and then dehydrated in an ethanol series. Thirty μl of probe mix were added and sealed under a 24×50 mm cover slip. Chromosomal and target DNAs were denatured together by incubating on a slide warmer at 65° C. for 30 seconds, and then transferred to 37° C. overnight for hybridization. Following hybridization, slides were washed at 45° C. for 20 minutes in 50% formamide/2×SSC, two times 10 minutes in 1×SSC and two times 10 minutes in 0.5×SSC. Slides were then blocked for 15 minutes at 37° C. with 4×SSC/3% BSA, and signals detected with Rhodamine-coupled sheep anti digoxigenin FAB fragments (Boehringer Mannheim), or avidin-fluorescein DCS (Vector Labs). Following detection, slides were washed three times in 4×SSC/0.1% tritonX100 for 5 minutes each, and mounted/counterstained with Vectashield containing Dapi and/or Propidium Iodide (Vector Labs) and viewed on a Nikon Axioskop microscope with epifluorescence.

v) Chromosome Walking Using Canine BAC End Probes:

The development of a high density BAC contig map was primarily based on chromosome walking and PCR assay results. The BAC clones were obtained through library screening by hybridization and verified through PCR of derived Sequence-Tag Site (STS) markers. For the purposes of contig-extension, the outlying STS-PCR products from each side of the contig were selected for hybridization of the high density gridded filters of the library as described in Li et al. (1999), supra. STS markers were designed to each end of each BAC clone. BAC end sequences were first analyzed with BLAST to identify common dog repetitive elements. PCR primers for STS markers were designed in regions of unique sequence using the Primer3 program available on the website of the Whitehead Institute for Biomedical Research/MIT Center for Genome Research. Amplification parameters were: 95° C. for 5 min and 25 cycles of 94° C. 1 min, annealing at 55 to 60° C. (depending on Tm of primers) 1 min and 1 min extension at 72° C. followed by a final 5 min extension at 72° C. PCR products were analyzed on 1.5% agarose gels followed by staining in ethidium bromide solution.

vi) Polymorphic Marker Isolation and Genetic Typing in Canine and Genomic BAC Clones

Microsatellite markers were isolated using minilibraries constructed from selected genomic BAC clones. Briefly, BAC clones were triple digested with Dra I, Ssp I and EcoRV (Amersham) and the resulting digests ligated to pBluescript, transformed and plated on LB/Agar plates covered with a Duralose-UV (Stratagene) membrane. Following overnight growth in a 37° C. incubator, replica filters were made using a second duralose membrane, applying pressure and marking by puncture. Replica filters were transferred to LB/Agar plates allowed to grow, and then colonies were lysed in situ by alkaline lysis as follows: membranes were placed on Whatman paper wet with 10% SDS for 5 min, and then transferred to denaturing and neutralizing solutions for 5 minutes each, followed by soaking in 6×SSPE. DNA was then crosslinked using UV light, and washed in 2×SSPE/1% SDS. After, the membranes were hybridized with γ-³²PdATP radiolabeled (CA)15, (GAAA)8, (GAAT)8 and/or (GATA)8 oligonucleotides and washed in 1×SSPE/0.1% SDS and 0.1×SSPE/0.1% SDS (55° C. and 65° C. respectively for dinucleotide versus tetranucleotide probes). Plasmid DNAs were extracted from all positive colonies (Qiagen) and sequenced on an ABI 377 DNA sequencer using T3 and T7 primers. The program primer3 was used to design flanking primers on all sequence traces containing a repeat sequence longer than 10 compound repeats. Amplification and detection of the fragment length polymorphism was performed as described in Lin et al. (1997) Tissue Antigens 50, 507-520.

vii) STS Typing and Contig Building:

The majority of the STS markers were developed by direct sequencing of BAC clone ends with T7 and SP6 using an ABI 377 DNA sequencer and by designing PCR primers. Other STSs were developed as part of our effort to clone dinucleotide and tetranucleotide microsatellite repeat markers in the region. These markers were used to test all BAC clones. BAC clone insert sizes are determined using Not I digestion followed by pulsed field gel electrophoresis in 1% agarose with a CHEF-DRII system (BioRad) and as described in Li et al. (1999), supra. STSs for which location was not strictly constrained were spaced at roughly equidistant intervals between constrained markers. To verify clone integrity, fingerprinting was performed on all clones using EcoR V, Hind III and Bgl II. Fragment size were estimated after ethidium bromide staining using established molecular weight markers and the Biorad 200 imaging system. Contig assembly was performed manually with assistance of the contig ordering package [Whitehead Institute] and Segmap for STS mapping (Green et al. (1991) PCR Methods Appl. 1, 70-90) and FingerPrint contig (available on the Sanger Center website) for fingerprinting (Soderlund et al. (1997) Comput. Apll. Biosci. 13, 523-535).

viii) Bioinformatics

Sequence contig and sequence comparisons were performed using with Sequencher 3.0 program (Gene Codes). cDNA-genomic DNA comparisons were performed using the BLAST program (available on the NCBI website). Genemap 1996 (The Human Transcript Map) and Genemap 1998 can be also found on the NCBI website. The Sanger Center Human Chromosome Radiation Hybrid Maps are also available on the internet. The Stanford Human Genome Center RHServer can be used to submit sequences on the internet. The Whitehead physical mapping project can also be found on the “carbon” server on the internet. The FPC (Software for FingerPrinting Contigs) is available through the Sanger Center website. The human gene mutation database is available through the website of the Institute of Medical Genetics, Cardiff of the University of Wales College of Medicine.

ix) Linkage Analysis and Region of Initial Linkage in Canine Narcolepsy

Autosomal recessive transmission with full penetrance for canine narcolepsy was first established in Labrador retrievers and Doberman pinschers (Foutz et al., 1979, supra). A large number of backcrosses were generated in the late 1980s in preparation for a linkage study using randomly distributed markers and a candidate gene approach (Cederberg et al., 1998, supra). Using this approach, genetic linkage between canarc-1 and the canine Major Histocompatibility Complex was excluded (Mignot et al., 1991, supra). A tightly linked marker was later identified using a human μ-switch immunoglobulin variable heavy chain probe (Mignot et al., 1991, supra). This initial μ-switch-like linkage marker was cloned using a HaeIII-size selected library (Mignot et al. (1994) Sleep 17, S60-S67; Mignot et al. (1994) Sleep 17, S68-S76). Sequencing of the fragment revealed a GC-rich repetitive sequence with high homology to the human μ-switch locus but no single copy sequence. Further cloning and sequencing studies using a Sau IIIA1 partially digested canine genomic phage library failed to identify a neighboring immunoglobulin gene constant region. This result indicated that the μ-switch sequence was a cross-reacting repeat sequence of unknown significance rather than a genuine immunoglobulin switch segment.

Chromosome walking using phage and cosmid libraries was difficult because of the small sizes of inserts in available libraries. We therefore decided to build a large insert Bacterial Artificial Chromosome (BAC) canine genomic library for this purpose (Li et al., 1999, supra). The large insert canine genomic BAC library was built using EcoRI partially digested DNA fragments from a Doberman pinscher. An animal born in one of our backcross litters and heterozygous for canarc-1 was selected to build the library. Having both the control and narcolepsy haplotypes in separate BAC clones would allow us to identify all possible disease-associated polymorphisms, and thus the mutation. Approximately 166,000 clones were gridded on 9 high-density hybridization filters. Insert analysis of randomly selected clones indicated a mean insert size of 155 kb and predicted 8.1 fold coverage of the canine genome (Li et al., 1999, supra). A 1.8 Megabase contig (77 BAC clones) was built in the region in an attempt to flank the canarc-1 gene. BAC clones containing our μ-switch-like marker were isolated and chromosome walking initiated from the ends. Microsatellite markers were developed in the contig and 11 polymorphic markers typed in all informative animals. (GAAA)ₙ repeats (rather than most typically used (CA)ₙ repeats) were found to be the most informative repeat markers in canines as previously reported (Ostranderet al. (1995) Mamm. Genome 6, 192-195; Francisco et al. (1996) Genome 5, 359-362). All informative animals, whether Dobermans or Labradors, were concordant for all the (CA)ₙ and (GAAA)ₙ repeat markers developed in this contig. The absence of any recombination events in this interval made it impossible to determine the location of canarc-1 in relation to our contig.

b) Results

The following provides a description of the results obtained in experiments that lead to the identification of the narcolepsy susceptibility locus (subsequently identified as a hypocretin receptor polymorphism) in a canine model of narcolepsy.

i) Homology Mapping Between Human Chromosome 6 and Canine Chromosome 12

BAC end sequence data obtained during through chromosome walks was analyzed with BLAST against appropriate Genbank databases. A BAC end sequence with high homology to Myo6, a gene located on the long arm of human chromosome 6 (6q12), was identified. A protocol for sequential G-Banding and canine chromosomal Fluorescence In-Situ Hybridization (FISH) was established (Li et al., 1999, supra). Briefly, DNA from the DLA locus was labeled with biotin and detected with avidin FITC, DNA from a canine BAC clone containing the μ switch-like marker and the Myo6 gene was labeled with digoxigenin and detected with anti-digRhodamine as described in (Li et al., 1999, supra). Both DLA (Dog Leukocyte Antigen), the canine equivalent of HLA (6p21), and BAC clones from the contig described here were found to be on canine chromosome CFA12 but at a very large genomic distance (>30 Mb). The dog autosomes were all acrocentric. Note that although the published localization of DLA is the telomere of CFA12 (Dutra et al. (1996). Cytogenet. Cell Genet. 74, 113-117), the result obtained here demonstrates a localization of DLA to the centromere of CFA12.

The results from the FISH analysis caused us to suspect a large region of conserved synteny between human chromosome 6 and canine chromosome 12. This large region of conserved synteny has been reported by other investigators [dog chromosome 12 is also called U10 based on radiation hybrid data] (Wakefield et al. (1996) Mamm. Genome 7, 715-716; Neff et al. (1999) Genetics 151, 803-820; Priat et al. (1998) Genomics 54, 361-378; Ryder et al. (1999) Anim. Genet. 30, 63-5).

Homology mapping experiments were conducted to facilitate identification of the narcolepsy susceptibility region. Human Expressed Sequence-Tag clones (ESTs) known to map a few centimorgans distal and proximal to Myo6 were obtained and used as hybridization probes on the canine BAC library filters. Positive clones were analyzed using two color FISH on dog metaphase spreads to screen for clones mapping to this portion of CFA12. This novel strategy successfully identified approximately 150 canine BAC clones that were shown to contain the canine equivalents of their corresponding human ESTs through hybridization and sequence analysis of plasmid subclones (data not shown). Minilibraries from these clones were generated to develop dinucleotide and tetranucleotide polymorphic markers, which were typed in our canine crosses and unrelated narcoleptic dog founders. This process was successfully repeated using all available single copy ESTs mapping within the region in humans until the canine narcolepsy critical region was flanked (the more precise map position of several ESTs was first estimated using the Stanford G3 radiation hybrid panel in several cases). Chromosome walking by filter hybridization was also performed until the region was almost entirely physically cloned. FIG. 1 provides a schematic of the region containing the canine narcolepsy gene, with the human canine chromosomal regions of conserved synteny displayed. Physical distances in human were estimated by mapping the corresponding clones on the Stanford G3 radiation hybrid panel and using a rough estimated correspondence of 26 kb/cR.

Backcross breeding was continued in parallel with the physical cloning effort. A Doberman litter born in our colony yielded our first narcolepsy/immunoglobulin-like marker recombinant animal, which mapped the region proximal to the Prim2A locus (“DC”, see FIG. 1). This finding, together with the observation of a crossover immediately distal to EST 858129 (“Ringo”, FIG. 1), reduced the narcolepsy susceptibility interval to an estimated 4 Mb region (EST 858129 to Prim2A in FIG. 1) in a total of 100 informative backcross animals. Two pedigrees identified in outside breeder colonies were used to further reduce the segment. The first pedigree is a familial narcolepsy Dachshund litter with 3 affected and 2 unaffected animals (NY, USA). Linkage with the canarc-1 locus was considered likely in this litter, considering previously established linkage of this region in other breeds. A maximum LOD score of 2.0 at 0% recombination (p=0.01) was obtained in this litter for the region immediately proximal to and including the Hcrtr2 locus (all animals concordant). This Dachshund pedigree includes a recombinant asymptomatic animal “Fritz” (FIG. 1). The second pedigree is a very large Doberman breeder pedigree (NJ, USA) with 7 affected animals. One of the affected animals was donated to the colony and shown to be canarc-1 positive by breeding. In this pedigree, all narcoleptic animals are identical by descent in a region flanked proximally by EST 250618 (Jayde and Tasha, FIG. 1). These findings allowed us to narrow down the canine narcolepsy susceptibility region to a subsegment of approximately 800 kb flanked by EST 250618 and Hcrtr2.

The distance between the initial linkage marker and the critical region corresponded to a 10 cM distance on the human map (FIG. 1) within an extensive region of conserved synteny. However, the canine genetic distance estimated from the breeding studies described here indicates that the distance is 1 cM (only one recombinant animal, “DC”, over 100 backcross animals). It is suspected that the region syntenic to the human chromosome centromere may have repressed recombination for an unknown reason. A map of the region as currently characterized is depicted in FIG. 1. The EST 858129 to Prim2A segment is approximately 4 Mb in humans (FIG. 1) as estimated through radiation hybrid data (3 cM on the human map). Interphase and metaphase FISH data in canines indicate the region is approximately of the same physical size in canines (data not shown). A small gap (estimated at 400 kb, based on the human radiation hybrid data, canine clone contig size, and canine FISH data) remains in the contig, between the hypocretin receptor 2 (Hcrtr2) and procollagen alpha2 IV genes (FIG. 1). The precise location of the canine narcolepsy gene is between EST 250618 and a region immediately distal to the hypocretin receptor 2 gene between markers 26-12 and 530-5 (FIG. 2). The estimated overall LOD score in the critical region is 32.1 at 0% recombination (n=105 animals) (Ott (1991). Analysis of Human Genetic Linkage. (Baltimore: Johns Hopkins University Press). Twenty-five dogs born in the NJ breeder colony were not included in the calculation due to inbreeding loops, missing animals and the difficulty in establishing precise family relationships in some cases.

### Example 2

### Identification of a Restriction Fragment Length Polymorphism (RFLP) in the Vicinity of the Hypocretin Receptor 2 Gene

Only one previously identified gene, Hcrtr2, was known to reside within the critical region identified in Example 1. This gene encodes a G-protein coupled receptor with high affinity for the hypocretin neuropeptides. To explore the possibility of an involvement of Hcrtr2 in the etiology of canine narcolepsy, BAC clones containing either the canarc-1 or the wild-type associated haplotypes were identified using previously identified polymorphic markers (see FIG. 2).

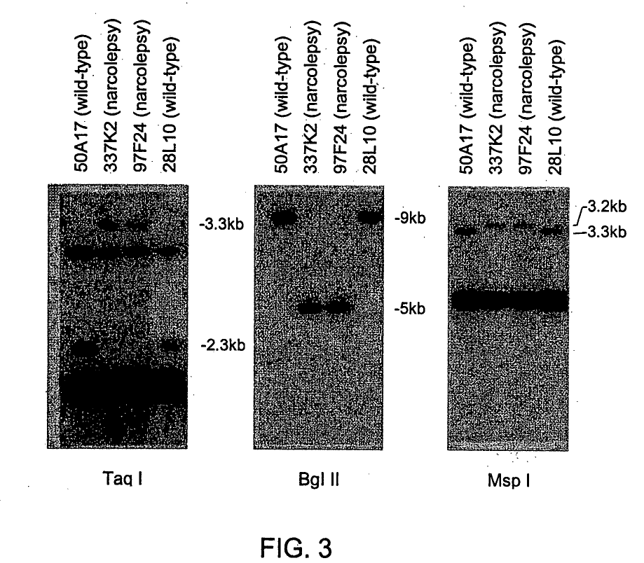

Narcolepsy (337K2, 97F24) and control (50A17, 28L10) allele associated BAC clones containing the canine homolog of the HCRTR2 gene were digested with four enzymes (Hind III, Bgl II, Taq I, Msp I), electrophoresed, transferred to nylon membrane and hybridized with a human hypocretin receptor 2 EST probe (IMAGE clone 416643 (HCRTR2)). A clear Restriction Fragment Length Polymorphism (RFLP) pattern was observed with three of the four enzymes (Bgl II, Taq I, Msp I) indicating a genomic alteration in the vicinity of or within the canine Hcrtr2 gene (FIG. 3). Hind III digest showed no restriction length polymorphism (data not shown).

### Example 3

### Canine Narcolepsy is Caused by a Mutation in the Hypocretin Receptor 2 Gene

With the above as guidance, PCR was performed to further characterize the polymorphism associated with narcolepsy. Briefly, total RNA extraction and mRNA purification from wild-type (4 Dobermans, 2 Labradors) and narcoleptic (4 Dobermans, 2 Labradors) dog brain were performed using the Rneasy Maxi (Qiagen) and Oligotex mRNA Midi Kits (Qiagen) respectively. First-strand cDNA was generated using mRNA (1 μg), AMV reverse transcriptase (SuperScript II RT; 200U; GIBCO BRL) and E. coli RNaseH (2U) according to the manufacturer's recommendation. PCR primers and conditions for RT-PCR amplification are described below. The PCR products were then sequenced and the resulting sequences compared with normal sequence to identify narcolepsy-causing mutations. Specific PCR amplification experiments are described in more detail below:

a) PCR of Wild-Type and Narcoleptic Doberman DNA using 5′ and 3′ Hcrtr2 Sequences

Degenerate consensus primers were designed based on the 5′ and 3′ sequences of the published human and rat Hcrtr2 cDNAs. Briefly, cDNAs were prepared from the brains of 4 control and 4 narcoleptic Dobermans born in the dog colony using one of three different sets of PCR primers. A first set (results shown in FIG. 4A) were designed in the 5′ and 3′ untranslated regions of the HCRTR2 gene (exon 1 and exon 7). The forward PCR primer was of the sequence: 5-2 (5′GCTGCAGCCTCCAGGGCCGGGTCCCTAGTTC 3′ (SEQ ID NO:1)); and the reverse primer was of the sequence: 3-2 (5′ATCCCTGTCATATGAATGAATGTTCTACCAGTTTT 3′ (SEQ ID NO:2)). As shown in FIG. 4A, the amplification product from the control dog (Lane 1) is the expected 1.6 kb size, whereas the product from narcoleptic dog (Lane 2) is 1.5 kb.

Amplified products from the cDNA of narcoleptic dogs significantly differed in size from the products of the controls (1.5 versus 1.6 kb). This finding indicated a deletion in the transcripts of narcoleptic animals (FIG. 4A). Sequence analysis of the RT-PCR product in narcoleptic and control animals indicated a 116 bp deletion, a result also confirmed by nested PCR experiments on c-DNA templates (data not shown).

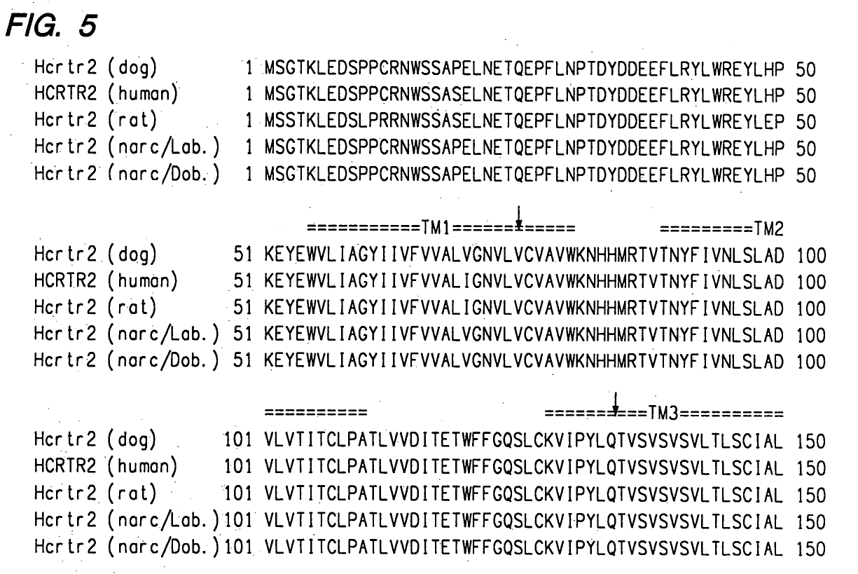

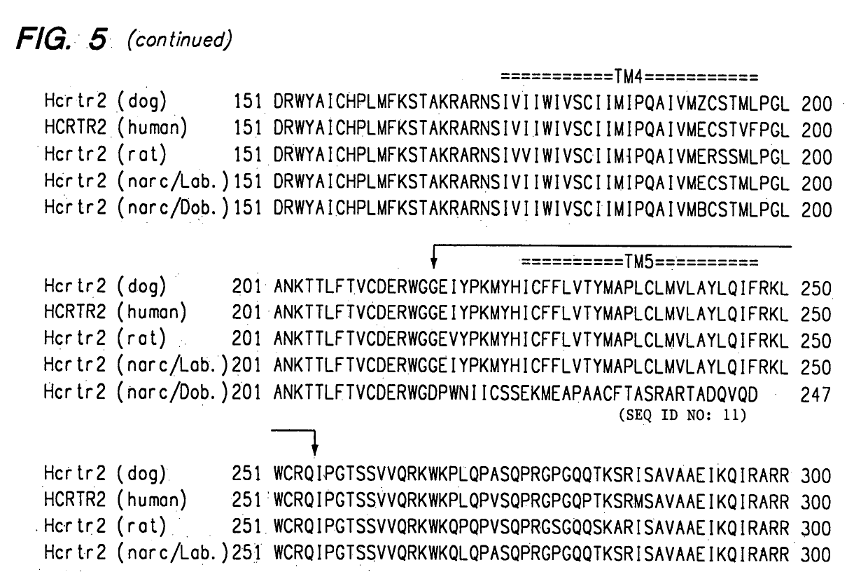

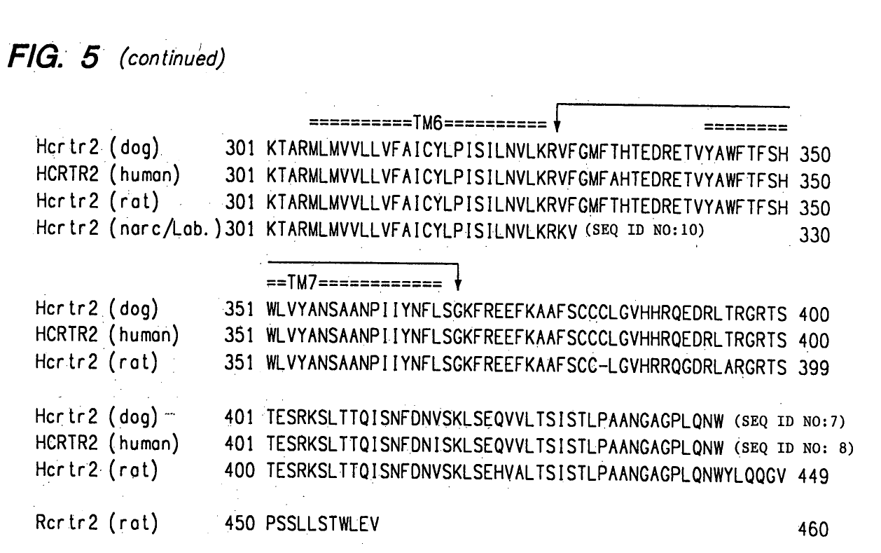

PCR primers scattered throughout the entire coding sequence were used to directly sequence the corresponding BAC clones representing both control and narcoleptic haplotypes. This allowed us to determine exon-intron boundary sequences of the locus in control and mutant alleles. The amino acid sequences of the corresponding Hcrtr2 of a wild-type dog, a narcoleptic Labrador, and a narcoleptic Doberman are aligned in FIG. 5.

The 166 bp deletion in the Hcrtr2 transcript corresponds to the exon 4 (continuous line between arrowheads). Genomic sequencing of the intron-exon boundary immediately preceding this intron indicated that a 226 bp canine short interspersed nucleotide element (SINE) (Minnick et al. (1992) Gene 110, 235-238; Coltman et al. (1994). Nucleic Acids Res. 22, 2726-2730) was inserted 35 bp upstream of the 3′ splice site of the fourth encoded exon (FIG. 6). This insertion falls within the 5′ flanking intronic region needed for pre-mRNA lariat formation and proper splicing. The efficiency of pre-mRNA splicing is strongly affected by alterations of the site within the intron that binds to the U2 small nuclear RNP. This region of complementarity includes the branchpoint sequence (BPS) at the site of lariat formation (Reed (1985) Cell 41,95-105; Reed et al. (1988) Genes Dev. 2, 1268-76). In mammals the BPS is a poorly conserved element that conforms to a very loose consensus sequence (PyXPyTPuAPy) in which the adenine residue is of primary importance. The BPS is typically located between 18 and 40 nucleotides upstream of the 3′ splice junction, but this position may also vary considerably. Despite the loose constraints on the consensus sequence and relative position of the BPS, alterations in the sequence may nearly abolish splicing (Reed et al., 1985 and 1988, supra).

b) PCR Using Primers Flanking the SINE Insertion

In a second experiment, narcoleptic and wild-type Doberman Pinscher genomic DNA was amplified with PCR primers flanking the SINE insertion. The forward primer w554-65seqF (5′GGGAGGAACAGAAGGAGAGAATTT3′ (SEQ ID NO:3)) was located in intronic sequence upstream of the insertion. The reverse primer R4/7-6R(110) (5′ATAGTTGTTAATGTGTACTTTAAGGC3′ (SEQ ID NO:4)) was located in intronic sequence downstream of exon 4. PCR conditions were 95° C. for 2 min; 30 cycles of 94° C. for 1 min, 55° C. for 1 min, 72° C. 1 min.

As shown in FIG. 4B, a 419 bp amplification product was produced from DNA of wild-type dogs and a 645 bp product from narcoleptic Doberman Pinscher DNA. Products of both sizes are amplified from the DNA of Dobermans known to be carriers of narcolepsy, and also display prominent heteroduplex bands. FIG. 4B, Lanes 1-2: wild-type Dobermans (Alex and Paris); lanes 3-4: narcoleptic Dobermans (Tasha and Cleopatra); lanes 5-6: heterozygous carrier Dobermans (Grumpy and Bob).

The SINE insertion may thus have moved the functioning branchpoint sequence beyond the acceptable range for efficient splicing (illustrated in FIG. 6). PCR primers were designed in the immediate flanking area and PCR analysis performed in control and canarc-1 positive narcoleptic dogs of three breeds (Dobermans, Labradors and Dachshunds). This PCR analysis identified the same SINE insertion in 17 narcoleptic Dobermans, including 6 dogs not known to be related by descent by at least 4 generations but likely to be identical by descent as a result of a founder effect. The SINE insertion was not found in 36 control dogs including 14 Dobermans, 13 Labradors and 9 Dachshunds (FIG. 4B). Based on this result and the associated cDNA analysis, we conclude that the SINE insertion mutation is the cause of narcolepsy in Dobermans. Similar retrotransposon-insertion mutations have been reported to cause human disease (see Kazazian et al. (1999) Nature Genet. 22, 130, and the human gene mutation database available over the internet through the UWCM.

c) PCR of Narcoleptic and Wild-Type Labradors Using Primers Based on Hcrtr2 Sequence

The SINE insertion was not observed in canarc-1 positive animals from other breeds (3 Labrador retrievers and one Dachshund; data not shown), suggesting that other mutations in the Hcrtr2 gene might be involved in these cases. Hcrtr2 was amplified from narcoleptic and wild-type Labrador retriever cDNAs. Genomic DNA was amplified with PCR primers flanking exon 6 and intron 6 using 6INF(162) (5′GACTTCATTTGGCCTTTGATTTAC3′ (SEQ ID NO:5)) and 7EXR(1620) (5′TTTTGATACGTTGTCGAAATTGCT3′ (SEQ ID NO:6)). PCR conditions were 94° C. for 2 min; 5 cycles of 94° C. for 1 min, 58° C. for 1 min, 72° C. 1 min; 30 cycles of 94° C. for 1 min, 55° C. for 1 min, 72° C. 1 min; 72° C. 5 min. Cycle sequencing on the PCR product was performed using the 6INF(162) primer and reactions analyzed on an ABI 377 DNA sequencer.

As shown in FIG. 4C. the amplification product from the control dog (Lane 1) is the expected 500 bp size, whereas the product from narcoleptic dog (Lane 2) is 380 bp. RT-PCR analysis was performed using c-DNAs prepared from the brains of 2 control and 2 narcoleptic Labrador retrievers born in our colony. Dachshund cDNA samples were not studied as no brain samples were available. A shorter PCR product was observed in narcoleptic versus control Labrador retrievers (FIG. 4C).

Sequencing indicated a deletion of exon 6 (123 bp) in the narcolepsy-associated cDNA. Analysis of the intron-exon boundaries and sequencing of exon 6 revealed a G to A transition in the 5′ splice junction consensus sequence (position +5, exon 6-intron 6) in genomic DNA of narcoleptic Labrador retrievers (FIG. 6). This G to A transition was not observed in the corresponding sequences of 24 control dogs (11 Labradors, 10 Dobermans, 3 Dachshunds), and 11 non Labrador narcoleptic dogs (10 Dobermans and 1 Dachshund). The consensus position for the +5 nucleotide is G (84%) and an A in this position is rarely observed (Shapiro et al. (1987) NuclAcids Res. 15, 7155-7173; Krawczak et al. (1992) Hum. Genet. 90, 41-54). A G to A transition reduces the likelihood functional score for the 8 nucleotide splicing consensus sequence from 88.4 to 74.8% (Shapiro et al., 1987, supra). Mutations in this position have been shown to produce 100% exon skipping (Krawczak et al., 1992, supra; McGrory et al. (1999) Clin. Genet. 55:118-121; Teraoka (1999) Am. J. Hum. Genet. 64, 1617-1631).

The Hcrtr2 transcripts produced in narcoleptic animals are grossly abnormal mRNA molecules. In Doberman pinschers, the mRNA potentially encodes a protein with 38 amino acids deleted within the 5th transmembrane domain followed by a frameshift and a premature stop codon at position 932 in the encoded RNA. The protein encoded by narcoleptic Labradors is also truncated at the C terminal and does not include a 7th transmembrane domain. These changes most likely disrupt proper membrane localization and/or cause loss of function of this strongly evolutionary conserved protein. These mutations are consistent with the observed autosomal recessive transmission of the disorder in these breeds.

### Example 4

### Hypocretin Levels in Cerebrospinal Fluid Correlate with Narcolepsy in Humans

In order to test whether a disruption in hypocretin neurotransmission causes human narcolepsy, hypocretin levels were assessed in volunteer narcoleptic and control (non-narcoleptic) subjects recruited in the Department of Neurology at Leiden University. Details of each patient's age, sex, Multiple Sleep Latency Test results, presence of cataplexy, duration of illness, and current pharmacological treatment are provided in Table 1. Hypocretin levels were measured in the cerebrospinal fluid (CSF) obtained by lumbar puncture of 9 narcoleptic (48.6±4.8 years [mean±SE]; 4 females) and 8 control (40.3±4.7 years; 5 females) subjects. All narcoleptic patients exhibited definite narcolepsy-cataplexy and were HLA DR2/DQB1*0602 positive (see Table 1). Samples were immediately frozen, coded and shipped blindly to Stanford University. Hypocretin was extracted from 1 ml of CSF (second fraction of 1.5 ml) using a reversed phase SEP-PAK C18 column. A ¹²⁵I hypocretin-1 radioimmunoassay (Phoenix peptide, Mountain View, Calif.) was used to measure levels in reconstituted aliquots (duplicates for each sample). Results are presented in Table 1.

Hypocretin-1 was detectable in all control subjects, with little inter-individual variation (ranging from 250 to 285 pg/ml) (Table 1). In 7 of 9 patients however, hypocretin levels were below the detection limit of the assay (<40 pg/ml) (p<0.007, Mann-Whitney U test). Undetectable levels were observed in both medicated and unmedicated patients, and were not associated with age, sex nor duration of illness (Table 1). Two subjects with an unquestionable diagnosis of narcolepsy-cataplexy (patients #4 and 5 in Table 1) had normal and elevated levels respectively. In Table 1: n.a.=not applicable; MSLT=Multiple Sleep Latency Test; SL and SOREMP=Mean Sleep Latency and number of Sleep Onset REM Periods in 5 or 4 (marked by *) naps. All CSF examinations (cell counts, protein and glucose levels) were within normal range. Recovery rate for the extraction of hypocretin-1 was 60.2∀3.8 (% ∀SD), and intra-assay variability for the measurement (extraction and RIA) was 3.8%. All samples were measured twice with comparable results.

TABLE 1
CSF hypocretin-1 levels and clinical features of narcoleptic and control
subjects.

| | | | | | | Duration | | |
|---|---|---|---|---|---|---|---|---|
| | Age | | MSLT | | | of illness | Current pharmacological | Hypocretin-1 |
| Subjects | (yrs) | Sex | SL(min) | SOREMPs | Cataplexy | (yrs) | treatment (daily dose) | (pg/ml) |
| Patients | | | | | | | | |
| 1 | 27 | m | 1.0* | 3* | + | 9 | GHB 5.6 g/methylphenidate | <40 |
| | | | | | | | 5-10 mg | |
| 2 | 34 | m | 0.9 | 5 | + | 4 | untreated for 2.5 months | <40 |
| 3 | 39 | f | 2.0* | 2* | + | 1 | Clomipramine 10 mg | <40 |
| 4 | 45 | f | 3.0 | 2 | + | 14 | Methylphenidate 30 mg | 255 |
| 5 | 50 | m | 6.3* | 3* | + | 19 | Clomipramine 30 mg/GHB | 638 |
| | | | | | | | 3.0 g | |
| 6 | 50 | m | 1.2 | 3 | + | 32 | GHB 5.4 g/modafinil 400 mg | <40 |
| 7 | 53 | f | 1.2 | 1 | + | 19 | GHB 4.0 g | <40 |
| 8 | 69 | f | 2.8 | 2 | + | 38 | Clomipramine | <40 |
| | | | | | | | 10 mg/modafinil 200 mg | |
| 9 | 70 | m | 2.1 | 2 | + | 53 | untreated for 20 years | <40 |
| Controls | | | | | | | | |
| 1 | 22 | m | na | na | − | na | — | 285 |
| 2 | 23 | f | na | na | − | na | — | 285 |
| 3 | 33 | m | na | na | − | na | — | 250 |
| 4 | 45 | m | na | na | − | na | — | 280 |
| 5 | 45 | f | na | na | − | na | — | 280 |
| 6 | 46 | f | na | na | − | na | — | 285 |
| 7 | 48 | f | na | na | − | na | — | 280 |
| 8 | 61 | f | na | na | − | na | — | 285 |

These data demonstrate for the first time that hypocretin neurotransmission is deficient in most cases of human narcolepsy. These results, particularly when combined with the observation that hypocretin receptor and peptide gene alterations induce narcolepsy in animal models, strongly support the conclusion that the hypocretin deficiency demonstrated in patients with undetectable levels causes narcolepsy. In contrast to the animal models, however, human narcolepsy is rarely familial and typically involves environmental factors on an HLA susceptibility background (Mignot (1998). Neurology 50, S16-S22). Without being held to theory, the decreased hypocretin neurotransmission in these patients is thus not likely to be due to highly penetrant hypocretin mutations. Rather, narcolepsy in these patients likely results from an HLA associated autoimmune-mediated destruction of hypocretin-containing neurons in the lateral hypothalamus.

The two patients with normal (255 pg/ml) and elevated (638 pg/ml) levels were both HLA-DQB1*0602 positive and clinically undistinguishable from the other narcoleptic patients. One explanation involves receptor/effector-mediated deficiency (as opposed to a defect in hypocretin production). Indeed, hypocretin-1 levels are detectable in the CSF of hypocretin receptor-2 mutated Dobermans (narcoleptic, n=33, 273.5±5.8 [mean±SE] pg/ml, control, n=9, 258.0±6.6 pg/ml, unpublished data). The considerably high hypocretin levels observed in patient #5 may also indicate an upregulation of hypocretin-1 production.

The above data further support a role for hypocretins in regulation of sleep patterns, with narcolepsy being an extreme form of improperly regulated sleep. Hypocretin neurons are discretely localized in the lateral hypothalamus, but have diffuse projections (Peyron, et al. 1998, supra). Of special interest are the dense projections to monoaminergic cell groups and the excitatory nature of this neuropeptide (Peyron, et al. 1998, supra). Hypocretin deficiency may decrease monoaminergic tone, an abnormality previously suggested to underlie the narcolepsy symptomatology, and could explain the beneficial effect of currently prescribed narcolepsy treatments (Nishino, et al. (1997), supra).

The results above also indicate that detection of hypocretin levels in the CSF is useful in the diagnosis of narcolepsy. The relative consistency of hypocretin levels between normal (non-narcoleptic) subjects, as well as a high incidence of decreased hypocretin levels in narcoleptic affected subjects, makes hypocretin a good diagnostic marker (e.g., to facilitate diagnosis of narcolepsy in a subject).

### Example 5

### Narcolepsy-Cataplexy in Humans Caused by Hypocretin Mutations

In contrast with the canine model, human narcolepsy is not a simple Mendelian disorder (Mignot 1998, supra). Human narcolepsy is HLA-associated, with more than 85% of patients with definite cataplexy carrying the HLA-DQB1*0602 allele. This finding led to the proposal that narcolepsy may be an autoimmune disorder. Twin studies indicate an important role for environmental triggers in the development of narcolepsy since only 25-31% of monozygotic twins are concordant for narcolepsy. Familial aggregation studies indicate a 20-40 fold increased genetic predisposition in first degree relatives but genuine multiplex families are rare. HLA-DQB1*0602 association is much lower in multiplex families than in sporadic cases, suggesting the existence of additional non-HLA genetic factors (Mignot (1998), supra).

In order to investigate the role of polymorphisms in human narcolepsy, exons and associated flanking intronic regions of the HCRT, HCRTR1 and HCRTR2 loci were sequenced in a pool of 70 narcoleptic and 152 control Caucasian subjects. To maximize the likelihood of finding mutations, the pools included subjects with and without the HLA-DQB1*0602 marker, as well as and subjects with and without a family history from the Stanford narcolepsy patient database. All patients had cataplexy, the clinical hallmark of the disorder (Aldrich 1998, supra). Eighty percent of these subjects had undergone nocturnal polysomnography and Multiple Sleep Latency Testing (MSLT) showing abnormalities diagnostic of narcolepsy (MSLT mean sleep latency ≦8 min, ≦2 Sleep Onset REM Periods [SOREMPs]).

To determine exon-intron boundaries and flanking sequences of the HCRTR1 gene, lambda clones were isolated from a human genomic phage library (Clontech) using the human HCRTR1 cDNA as a probe. Positive phage clones were subcloned, and sequenced using an ABI 377 automated sequencer (PE Biosystems). HCRTR2 containing BAC clones 106-C-7, 575-E-23 and 575-M-3 were identified through PCR screening of BAC superpools (Research Genetics) using primers expected to amplify exons 1 and 7, based on published canine splice positions (Lin et al. (1999) Cell 98:365-376). Exon-intron boundaries and flanking sequence of the HCRTR2 locus were determined by directly sequencing human BAC clones with primers directed to the cDNA sequence. HCRTR1 and HCRTR2 each have 7 coding exons and the positions of the splice junctions with respect to the protein sequence are conserved across species and receptor subtypes. The complete genomic sequence of the human HCRT gene has previously been published by Sakurai et al. (1999) J Biol Chem 274:17771-17776. PCR primers were designed to allow amplification and sequencing of at least 50 bp flanking each exon of each of the three genes to identify coding alterations and mutations affecting mRNA splicing. Amplification products were purified using Qiaquick 96 PCR purification kits (Qiagen) and sequenced using BigDye sequencing mix (PE Biosystems). Reactions were column-purified (Edge Biosystems) and sequenced on an ABI 377. Sequence alignments and trace comparisons were performed using Sequencher 3.1 (Gene Codes).

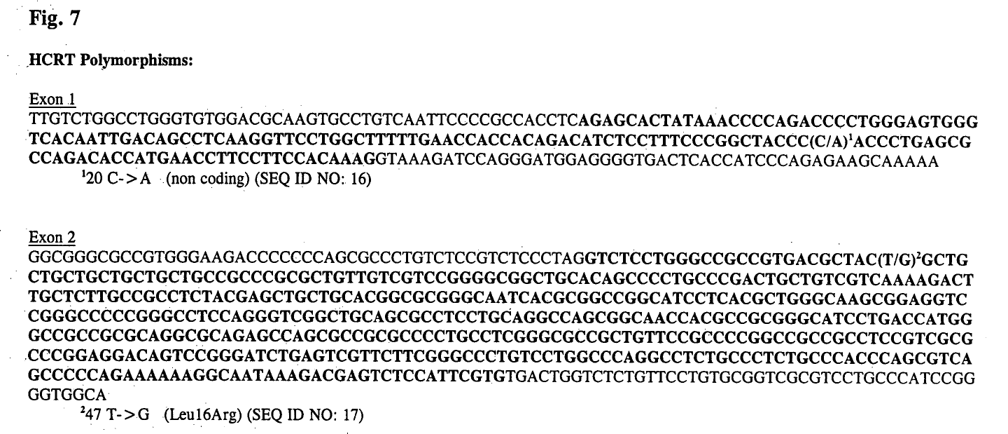

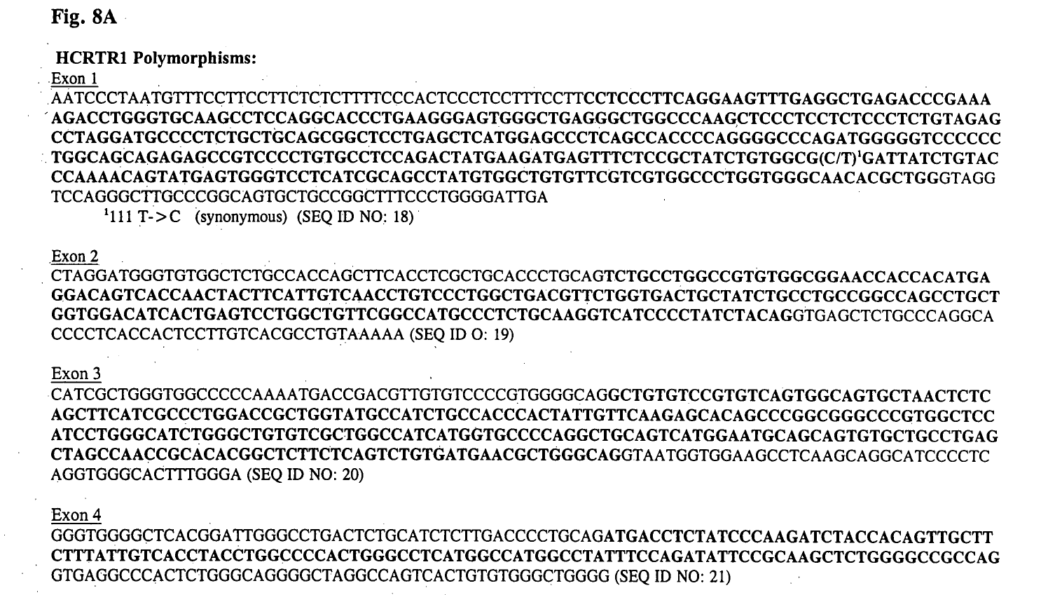

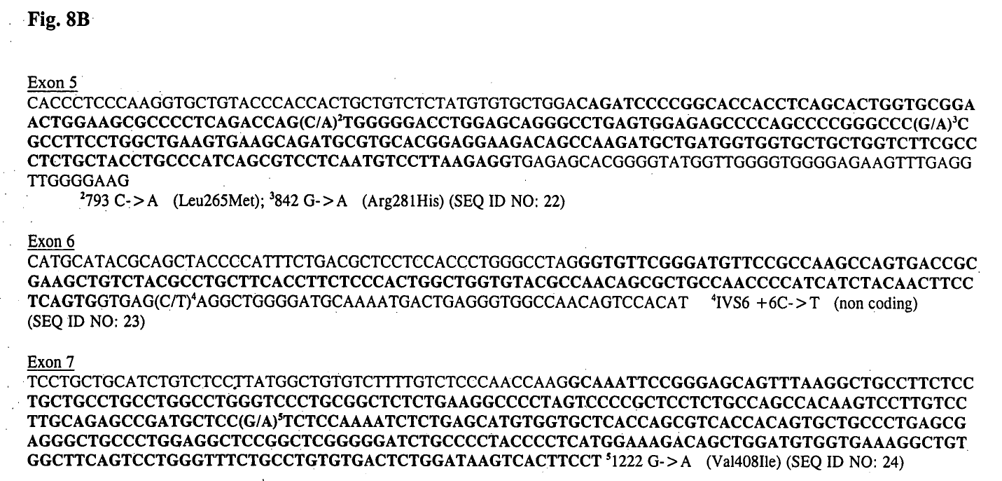

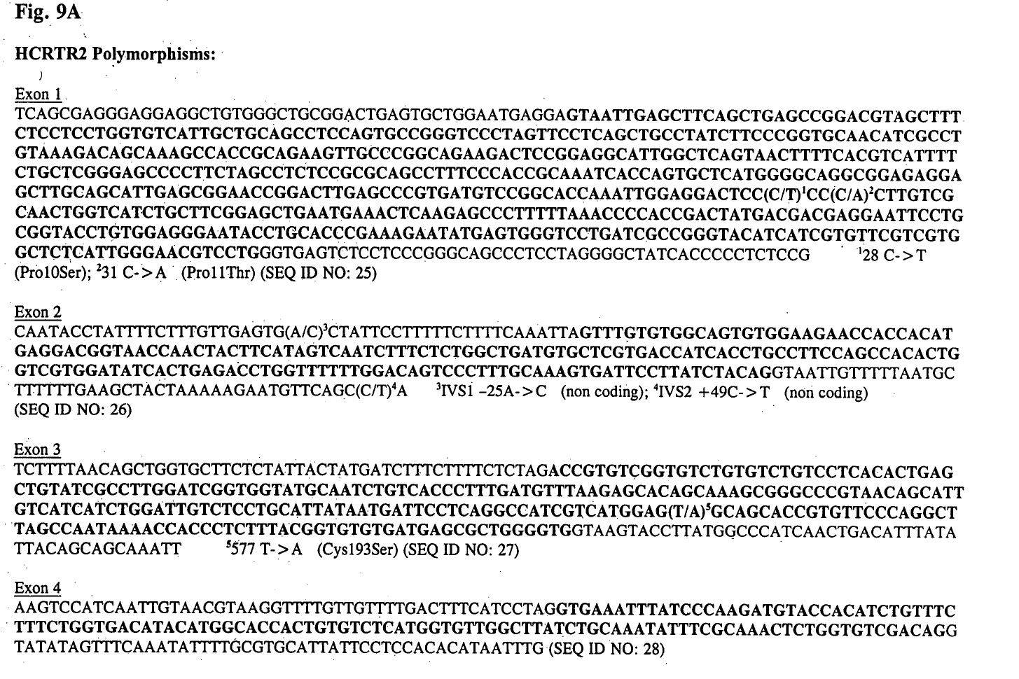

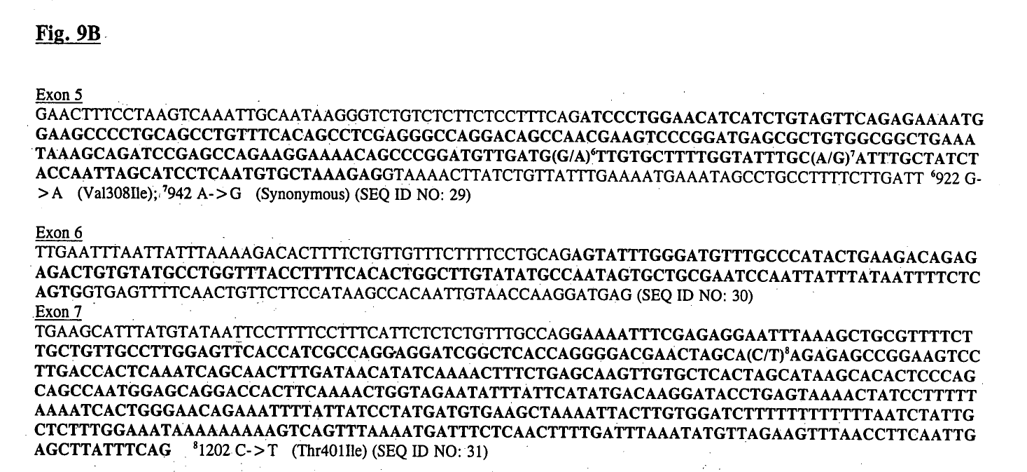

Fifteen polymorphisms were found. The details of each polymorphism are provided in Table 2. The DNA sequences of the native hypocretin peptide (HCRT), the hypocretin receptor 1 (HCRTR1), and the hypocretin receptor 2 (HCRTR2) are provided in FIGS. 7, 8a-8B, and 9A-9B, respectively, with each of the polymorphisms of the invention indicated. Exon sequences are in bold; flanking intronic sequences (approximately 50 bp of sequence on both sides of each exon) are also included. Polymorphic residues, if any, are indicated under brackets. HCRT (human hypocretin polypeptide gene) has two exons; HCRTR1 (human hypocretin receptor 1 gene) contains 7 exons; and HCRTR2 (human hypocretin receptor 2 gene) contains 7 exons. Sequencing of selected exons in additional control samples and family members indicated that most of these coding polymorphisms were not associated with narcolepsy (Table 1). In Table 2: DNA and amino acid changes are counted from the ATG-codon and Met-residue respectively;5′ untrans=5′ untranslated region; TM=transmembrane domain; I=intracellular loop IVS=intervening sequence (intron), position relative to adjacent exon; F+=familial, DQB1*0602 positive; F−=familial, DQB1*0602 negative; S+=sporadic, DQB1*0602 positive; S−=sporadic, DQB1*0602 negative.

TABLE 2
Allelic variance of the HCRT, HCRTR-1, and HCRTR-2 loci in narcoleptic and control subjects
Preprohypocretin (HCRT)

| | | | | | | | Control | |
|---|---|---|---|---|---|---|---|---|
| | | | | | | | allele | |
| | | | Narcolepsy | | | | frequency | |
| DNA | Amino acid | | allele frequency (number of subjects) | | | | (number of | |
| change | change | Domain | F+ | F− | S+ | S− | subjects) | Notes |
| −20C→A | non coding | 5′z untrans | 0.00 (15) | 0.00 (8) | 0.00 (22) | 0.056 (18) | 0.00 (15) | Presumed benign |
| | | | | | | | | polymorphism |
| 47T→G | Leu16Arg | signal | 0.00 (17) | 0.00 (8) | 0.00 (23) | 0.028 (18) | 0.00 (135) | Dominant mutation |
| | | peptide | | | | | | |

Hypocretin receptor 1 (HCRTR1)

| | | | | | | | Control | |
|---|---|---|---|---|---|---|---|---|
| | | | | | | | allele | |
| | | | Narcolepsy | | | | frequency | |
| | | | allele frequency (number of subjects) | | | | (number of | |
| DNA change | AA change | Domain | F+ | F− | S+ | S− | subjects) | Notes |
| 111T→C | synonymous | N-term | 0.30 (15) | 0.67 (6) | 0.33 (24) | 0.33 (16) | 0.36 (39) | Benign polymorphism |
| 793C→A | Leu265Met | I 3 | 0.00 (15) | 0.00 (6) | 0.02 (23) | 0.00 (17) | 0.00 (14) | Presumed benign |
| | | | | | | | | polymorphism |
| 842G→A | Arg281His | I 3 | 0.00 (15) | 0.00 (6) | 0.00 (23) | 0.00 (17) | 0.04 (14) | Benign polymorphism |
| IVS6(+6C→T) | non coding | introns | 0.06 (18) | 0.00 (7) | 0.02 (23) | 0.00 (17) | 0.08 (39) | Benign polymorphism |
| 1222G→A | Val408Ile | C-term | 0.27 (15) | 0.64 (7) | 0.33 (23) | 0.41 (17) | 0.34 (16) | Benign polymorphism |

Hypocretin receptor 2 (HCRTR2)

| | | | | | | | Control | |
|---|---|---|---|---|---|---|---|---|
| | | | | | | | allele | |
| | | | Narcolepsy | | | | frequency | |
| DNA | Amino acid | | allele frequency (number of subjects) | | | | (number of | |
| change | change | Domain | F+ | F− | S+ | S− | subjects) | Notes |
| 28C→T | Pro10Ser | N-terminus | 0.00 (17) | 0.00 (9) | 0.02 (23) | 0.00 (18) | 0.000 (90) | Presumed benign |
| | | | | | | | | polymorphism |
| 31C→A | Pro11Thr | N-terminus | 0.00 (17) | 0.11 (9) | 0.00 (23) | 0.00 (18) | 0.006 (90) | Unlinked with phenotype |
| IVS1 | non coding | intron | 0.13 (15) | 0.06 (8) | 0.18 (22) | 0.18 (17) | 0.18 (57) | Benign polymorphism |
| (−25A→C) | | | | | | | | |
| IVS2(+49C→T) | non coding | intron | 0.30 (15) | 0.25 (8) | 0.16 (22) | 0.26 (17) | 0.17 (58) | Benign polymorphism |
| 577T→A | Cys193Ser | TM IV | 0.00 (16) | 0.00 (9) | 0.00 (22) | 0.00 (17) | 0.01 (41) | Presumed benign |
| | | | | | | | | polymorphism |
| 922G→A | Val308Ile | TM VI | 0.12 (17) | 0.06 (8) | 0.20 (22) | 0.24 (17) | 0.19 (35) | Benign polymorphism |
| 942A→G | synonymous | TM VI | 0.06 (17) | 0.00 (8) | 0.00 (22) | 0.00 (17) | 0.01 (35) | Benign polymorphism |
| 1202C→T | Thr401Ile | C-terminus | 0.03 (17) | 0.00 (8) | 0.00 (22) | 0.00 (14) | 0.00 (96) | Possible weakly penetrant |
| | | | | | | | | allele in combination with |
| | | | | | | | | DQB1*0602 |

One case of narcolepsy was caused by a mutation in the HCRT locus. This patient is an HLA-DQB1*0602 negative patient with severe cataplexy (5-20 attacks per day when untreated), daytime sleepiness, sleep paralysis and hypnagogic hallucinations. HLA typing indicated DRB1*0402, DRB1*0701; DQB1*0202, DQB1*0302.

It is of particular interest that this patient first demonstrated cataplexy at 6 months of age. Most cases of human narcolepsy only appear during adolescence whereas narcolepsy in canines and knockout mice typically begins before sexual maturity (Mignot (1993) J. Neurosci. 13, 1057-1064; Mignot et al. (1993) Psychopharmacology 113, 76-82; Chemelli et al. (1999) Cell 98:437-451). SOREMPs were first documented during nocturnal sleep recordings at 3 years of age. Twenty four hour polysomnography at age 9 documented fragmented sleep/wake patterns and SOREMPs during sleep attacks. Interestingly, spike-slow wave complexes and low frequency (3-4 Hz) discharges without any associated clinical findings were also observed, mostly in combination with REM sleep. These findings are reminiscent of pre-REM sleep spindling activity reported in the preprohypocretin knockout mice (Chemelli et al 1999, supra). An MSLT performed at 11 years of age showed a mean sleep latency of 1.1 minutes and 4 SOREMPs. Additional clinical features include periodic leg movements poorly responsive to L-DOPA or clonazepam and episodic nocturnal bulimia since the age of 5. The patient is currently 18 years old and his symptoms are partially controlled with methylphenidate and either imipramine, clomipramine or fluoxetine.

The HCRT mutation in this subject is a valine to arginine substitution in the hydrophobic core of the signal peptide. The G−>T transversion responsible for the encoded arginine was not observed in 270 control chromosomes nor in the patient's unaffected mother (father unavailable). Signal peptide mutations are known to produce a variety of genetic disorders. The majority of these mutations display autosomal dominant transmission. These include familial isolated hypoparathyroidism (Arnold et al. (1990) J Clin Invest 86:1084-1087), autosomal dominant neurohypophyseal diabetes insipidus (Ito et al. (1993) J Clin Invest 91: 2565-2571), antithrombin deficiency (Fitches et al. (1998) Blood 92: 4671-4676), primary hypercholesterolemia (Cassenelli et la. (1998) Clin Genet 53:391-395) and chronic pancreatitis (Witt et al. (1999) Gastroenterology 117:7-10). Autosomal recessive inheritance has also been observed in a few cases such as Factor X deficiency (Santo Domingo type)(Watzke et al. (1991) J Clin Invest 88:1685-1689) and Crigler Najjar disease (Seppen et al. (1996) FEBS Lett 390:294-298). Functional analysis generally suggests dominant secretory dysfunction. In autosomal dominant neurohypophyseal diabetes insipidus, failure to cleave results in the accumulation of mutant polypeptides in the endoplasmic reticulum (Siggaard et al. (1999) J Clin Endocrinol Metab 84:2933-2941) and produces neurodegeneration as documented by Magnetic Resonance Imaging studies (Gagliardi et al. (1997) J Clin Endocrinol Metab 82:3643-3646). In hypoparathyroidism and hypercholesterolemia, the mutations place a highly charged arginine in the hydrophobic core of the signal peptide, as we observed in the HCRT precursor. The parathyroid hormone mutation results in a mutant polypeptide that has impaired translocation into the endoplasmic reticulum, and is poorly cleaved by signal peptidase (Karaplis et al. (1995) J Biol Chem 270: 1629-1635).

Another polymorphism of interest was observed in exon 7 of the HCRTR2 locus, causing a threonine to isoleucine substitution in the C terminal domain of the receptor. This substitution was observed in the proband of a multiplex family with two affected HLA-DQB1*0602 positive subjects, but was not observed among 192 control chromosomes. However, two unaffected relatives also carried the substitution in the pedigree. The presence of a hydroxylated amino acid (serine or threonine) is conserved in this position across species in both the HCRTR1 and 2 genes. This mutation could disrupt a phosphorylation site in the C terminal region of HCRTR2. Phosphorylation in the C-terminal area of other G-protein coupled receptors has been shown to mediate receptor desensitization (Ferguson et al. (1996) Can J Physiol Pharmacol 74:1095-1110; Gaudin et al. (1999) Biochem Biophys Res Comm 254:15-20) and disrupting this process could lead to dominant effects. Based on the pattern of inheritance we conclude that this substitution is probably benign but could act as a weakly penetrant narcolepsy susceptibility mutation in the presence of HLA-DQB1*0602.

These results demonstrate for the first time that hypocretin mutations in humans can produce the full narcolepsy phenotype, with definite cataplexy and other associated clinical features. This result validates previous work using animal models. It also indicates the implication of the hypocretin system in other human narcolepsy-cataplexy cases and describes hypocretin polymorphisms in humans that have potential applications in predicting treatment response and predisposition to other sleep, attention or mood disorders.

### Example 6

### Hcrt, But Not MCH, Transcripts are Absent in the Perifornical Area of Narcoleptic Patients

In order to examine the expression of the preprohypocretin mRNA in narcoleptic subjects, in situ hybridization studies were conducted—using a probe specific for the pre-prohypocretin gene. Expression of Melanin Concentrating Hormone (MCH), a peptide also expressed in the perifornical area of the human hypothalamus (Elias. et al. (1998) J Comp Neurol 402, 442-59), was examined as a control.

Brain tissue was isolated from narcoleptic and non-narcoleptic (control) human subject. Post mortem delays were 13.46 1.88 hrs (5 to 26 hrs) in controls and 24.6±15.2 hrs (4.5 to 98 hr narcoleptics. Coronal slices of brains (1 cm thick) including the entire hypothalamus region, the pons (locus coeruleus area) or the frontal cortex were immediately frozen on dry ice and stored at −80° C. Similar regions were used in control and narcoleptic subjects. Neuroanatomical experiments were conducted in 13 control subjects. Only 2 narcoleptic samples were found to contain the hypothalamus and were used for in situ hybridization. These 2 subjects were a 77 year old female with a postmortem delay of 6.75 hr and a 67 year old male with a postmortem delay of 17 hrs. Cryostat sections (15 μm thick) were made throughout the hypothalamus (from the mammillary bodies to the optic chiasm region), thaw-mounted onto poly-L-lysine coated slides and stored at −80° C.

Purified Hcrt and MCH oligodeoxynucleotides were provided by the PAN facility (Stanford, USA) or INTRON company (Kaltbrunn, Switzerland), re-suspended in ultra-pure water, aliquoted at 1 pmol/μl and stored at −20° C. Antisense probes for Hcrt and MCH were: S1HCRTHUM (bases 198-238) and S2HCRTHUM (bases 365-407) of the human prepro-Hcrt gene (GeneBank, NM_{—}001524); S1MCHHUM (bases 501-541) of the human pro-MCH gene (GeneBank, NM_{—}002674). Oligoprobes were 3′end labeled with [35S]-dATP (Amersham Pharmacia Biotech, Piscataway, N.J.) using a terminal deoxynucleotidyl transferase (Amersham Pharmacia Biotech) to a specific activity of at least 1×108 cpm/μg.). Oligonucleotides for human TNF-alpha (Oncogene Research Products, Boston, Mass.) were provided at 2.5 pmol/μl. Oligoprobes were 3′end labeled with [³⁵S]-dATP (Amersham Pharmacia Biotech, Piscataway, N.J.) using a terminal deoxynucleotidyl transferase (Amersham Pharmacia Biotech) to a specific activity of at least 1×10⁸ cpm/μg. Probes were purified on microspin G25 columns (Amersham Pharmacia Biotech). Corresponding sense oligoprobes were used as controls.

Coronal sections were thawed 30 min before being fixed in 4% Paraformaldehyde in 0.1M phosphate buffer (PBS) pH 7.4 for 10 min. After a 5 min rinse in 2× sodium chloride-sodium citrate buffer (SSC), slides were immersed in 0.1M Triethanolamine (pH 8) containing 0.25% of acetic anhydride for 10 min. They were then rinsed in 2×SSC for 5 min, dehydrated in ascendant concentrations of ethanol, delipidated for 10 min in chloroform and dipped in ethanol 100% and 95%. Sections were finally air-dried.

In situ hybridization were conducted as described in Charnay et al ((1999) J Chem Neuroanat 17, 123-8. Briefly, each section was hybridized with 1×10⁶ cpm of radiolabeled probe in 200 μl of hybridization buffer containing 50% deionized formamide, 4×SSC, 1× Denhardt's solution, 10% dextran sulfate, 10 mM dithiothreitol, 140 μg/ml yeast tRNA, 800 μg/ml denaturated salmon testes DNA and 100 μg/ml polyadenilic acid. The sections were coverslipped and placed at 42° C. overnight in a humid chamber. The slides were then washed in 1×SSC at 42° C. (2×30 min), 0.1×SSC at 42° C. (1×30 min), 0.1×SSC at room temperature (1×30 min), and 70% ethanol for 2 min to be finally air-dried. Signal was detected using beta-max autoradiographic hyperfilms (Amersham Pharmacia Biotech) for 8-10 days at 4° C. Sense oligoprobe and RNase pretreatment (30 min at room temperature) controls were conducted using adjacent sections.

Cell mapping was performed using a computerized image analysis system (Adobe Photoshop software) fitted to a camera (Kontron Progress 3008). The hypothalamic subdivisions were identified and named using the Mai et al.³⁷ atlas of the human brain. The total number of Hcrt mRNA expressing cells was estimated using a series of emulsion-coated sections taken every 100 μm along the entire hypothalamus of 2 subjects. Cell counts of radiolabeled cells were made under a Zeiss Axiophot microscope fitted to a computerized image analysis system (SAMBA, Alcatel, France).

Results

MCH mRNA expressing cells were more widely distributed than Hcrt positive cells, as previously reported(Peyron et al. (1998) J Neurosci 18, 9996-10015; Elias et al. supar; Broberger etla. (1998) J Comp Neurol 402, 460-74). Although partial overlap between MCH- and Hcrt-expressing cells was suggested, especially dorsal and dorsolateral to the fornix, the respective patterns of radiolabeling were generally distinct.

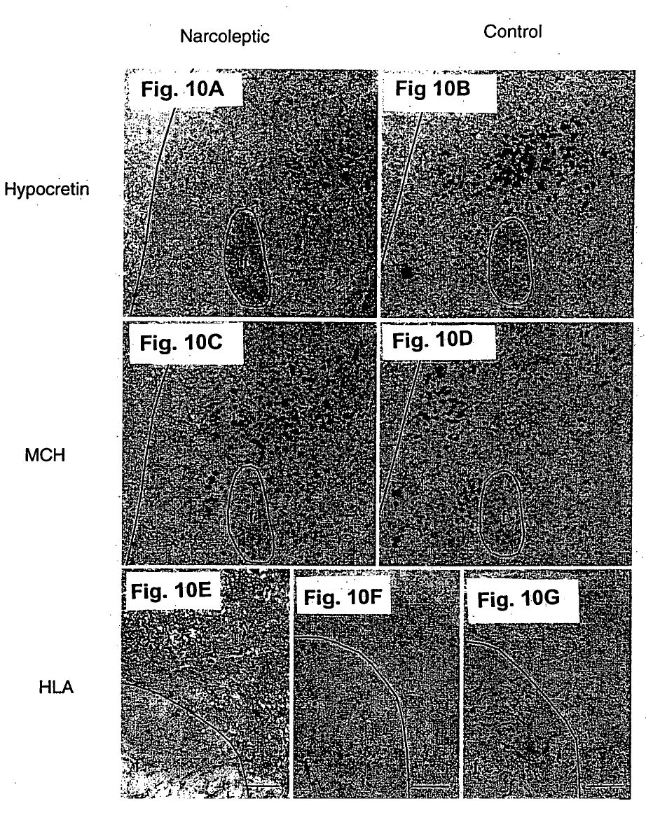

Hctr and MCH in situ hybridizations were next processed on adjacent sections in control and narcoleptic tissues. Sections from 4 controls and 2 narcoleptic subjects were processed in parallel. No signal for Hcrt was found in the hypothalamus of human narcoleptic subjects (FIG. 10A). In contrast, MCH neurons were observed on adjacent sections (FIG. 10C). In control tissues, both peptides were highly expressed (FIGS. 10B,D). MCH expression was similar in control and narcoleptic brains. Of note, both narcoleptic patients and 3 of 13 controls were HLA-DQB1*0602 and one narcoleptic subject had a family history for narcolepsy-cataplexy. These results demonstrate a lack of transcription in intact cells or a previous destruction of Hcrt-containing neurons.

### Example 7

### Hcrt-1 and Hcrt-2 Peptides are Undetectable in the Central Nervous System of Narcoleptic Subjects

Levels of Hcrt-1 and Hcrt-2 peptides in brain tissues from 8 control and 6 narcoleptic subjects were measured using radioimmunoassays. Two of the narcoleptic subjects and 4 of the controls were also used in the in situ hybridization study described in Example 6. Hcrt-1 and Hcrt-2 were measured using a commercially available RIA kit (Phoenix Pharmaceuticals, Mountain View, Calif.) containing anti-Hcrt-1 and ¹²⁵I Hcrt-1, or anti-Hcrt-2 and ¹²⁵I Hcrt-2, respectively. -Levels were determined using a standard curve (1-128 pg). Evaporated samples were re-suspended in 500 μl of RIA buffer. Recovery efficiency during extraction was determined using an internal standard (³H Hcrt-2, American Peptide, approx. 50,000 dpm [68 pmol]) and was found to be 58.3±2.5%. All reported values (pg/g of wet brain tissue) were adjusted to reflect the estimated original values before extraction. All measurements were conducted in duplicate using 10-100 μl of sample and in a single RIA. The intra-assay variability was 3.8%. The detection limit for Hcrt-1 and Hcrt-2 was 332 pg/g.

Results

All narcoleptic subjects had typical cataplexy and were HLA-DQB1*0602 positive. Three controls were HLA-DQB1*0602 positive. Peptide levels were measured in cortex (14 subjects) and available pons samples (4 subjects); these structures are known to receive hypocretin projections. Hcrt-1 and Hcrt-2 peptides were detectable in all control samples, independent of their DQB1*0602 status. Consistent with reports in rat brain (Mondal et al. (1999) Neurosci Lett 273, 45-8; Taheri etal. (1990), FEBS Lett 457, 157-61), hypocretin levels were 10-20 fold higher in the pons (Hcrt-1: 19,530 and 23,502 pg/g, and Hcrt-2: 12,109 and 14,571 pg/g) than in the cortex (mean±SEM, Hcrt-1: 939±239 pg/g; Hcrt-2: 1,561±323 pg/g). In the pons of 2 narcoleptic subjects, one of which was tested using in situ hybridization, Hcrt-1 and Hcrt-2 were well below control levels, in the undetectable range (<332 pg/g). Both peptide levels were also undetectable in cortex samples, with the exception of one subject with low cortical levels (Hcrt-1: 347 pg/g and Hcrt-2: 485 pg/g) and undetectable levels in the pons. These results confirm that Hcrt-1 and Hcrt-2 are absent in narcoleptic patients.

### Example 8

### Relevant Immunopathological Studies in the Perifornical Area Do Not Indicate Acute Inflammation or Extensive Neuronal Loss in the Region

The absence of hypocretin signal, together with the established HLA association in narcolepsy, suggests the possibility of an autoimmune destruction of Hcrt-containing cells in the hypothalamus. In order to test this hypothesis, coronal sections were stained with HLA Class II (HLA-DR) to examine the sections for evidence of inflammation and loss of neurons. Increased HLA-DR expression and microglial activation is a sensitive indicator of pathological events in the central nervous system (CNS) (Schmitt et al. (1998) Neuropathol Appl Neurobiol 24, 167-76).

HLA and Glial Fibrillary Acidic Protein (GFAP) immunostaining were performed on adjacent sections in the perifornical area. Frozen sections were air-dried for 30 min before being fixed with 4% paraformaldehyde-PBS 0.1 M, pH 7.4 for 20 min at room temperature. After 2 rinses in 0.1M PBS for 5 min each, sections were pre-incubated in bovine serum albumin (1:30 in PBS) for 1 hr at room temperature. Sections were incubated sequentially with a mouse anti-human DR-alpha antibody (1:100 in PBS; overnight at room temperature; clone TAL.1B5, Dako Corp., Carpinteria, Calif.) or a mouse anti-GFAP monoclonal antibody (1:500 in PBS, overnight at room temperature; Chemicon international Inc., Temeluca, Calif.), a biotinylated horse anti-mouse IgG (1:1000 in PBS; for 90 min at room temperature; Vector Labs. Inc, Burlingame, Calif.), and exposed to avidin-biotin-HRP complexes (1:1000 in PBS; for 90 min at room temperature; Vector Elite Kit, Vectastain). Sections were rinsed twice for 15 min in PBS after each incubation. The sections were immersed in 0.05 M Tris-HCl buffer, pH 7.6, containing 0.025% 3,39-diaminobenzidine-4HCl (Sigma, St. Louis, Mo.), 0.6% ammonium nickel (II) sulfate hexahydrate (Nacalai Tesque, Kyoto, Japan), and 0.003% H₂O₂, for 30 min at room temperature. The histochemical reaction was stopped using two rinses of PBS. After this procedure, microglia (HLA) or astrocytes (GFAP) were stained in black. Sections were blindly scored by 3 investigators as described in Tafti et al ((1996) J Neurosci 16, 4588-95).

Results

Thionin, crysal violet and GFAP staining of narcoleptic sections (n=2 subjects) revealed no obvious lesions or gliosis in the perifornical area. HLA-DR immunocytochemistry was performed in narcoleptic (n=2) and control tissues (n=4). The sections taken were adjacent to those used for Hcrt and MCH in situ hybridization experiments. Resting HLA-DR positive microglia were detected in the white and gray matter of control (FIG. 10G) and narcoleptic (FIG. 10E,F) subjects. Staining in the perifornical area was moderate and none of the cases were associated with activated, amiboid microglia. Microglial HLA labeling was higher in the white matter (fornix) than the gray matter (perifornical area), but did not differ between control and disease status (FIG. 10E-G). surprisingly, however, we also did not detect significant residual gliosis and/or cellular loss in the region. Further, MCH positive neurons were not affected by the disease process. In situ hybridization with Tumor Necrosis Factor (TNF)-alpha, a cytokine strongly expressed in many inflammatory CNS disorders, including multiple sclerosis and experimental autoimmune encephalomyelitis, also produced no significant signal in control and narcoleptic tissue.

Although autoimmune mediation for human narcolepsy has been suspected for since 1984, when the disorder was first shown to be associated with HLA-DR2. Further studies have established a tighter association with HLA-DQ, but no evidence for immunopathology has been found. In situ hybridization for TNF-alpha and immunocytochemistry for HLA reveal no sign of recent inflammation in the two brains examined. This might be explained by the fact that the 2 subjects were examined long after the Hcrt cells were putatively destroyed (more than 50 years after disease onset). More surprisingly, however, we also did not detect significant residual gliosis and/or cellular loss in the region. Further, MCH positive neurons were not affected by the disease process. This result is remarkable, considering that MCH and Hcrt-positive cells are intermingled in the region of interest. Hcrt-containing neurons are few in number (15-20,000 neurons), and dispersed within a limited area of the tuberal hypothalamus. This might explain the difficulty in detecting any overt lesion in histopathological studies.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for detecting a predisposition to a disorder in a subject caused by an alteration in hypocretin receptor activity, the method comprising:
analyzing nucleic acid of a subject for the presence of at least one polymorphism that predisposes the subject to a disorder caused by an alteration in activity of a hypocretin receptor;
wherein the presence of the predisposing polymorphism is indicative of an increased susceptibility of the subject to a disorder caused by an alteration in a hypocretin receptor activity.

2. The method of claim 1, wherein the predisposing polymorphism is in a hypocretin receptor gene.

3. The method of claim 1, wherein the predisposing polymorphism is in a hypocretin receptor-2 gene.

4. The method of claim 1, wherein the predisposing polymorphism is in a hypocretin polypeptide.

5. The method of claim 1, wherein the disorder is a sleep disorder.

6. The method of claim 5, wherein the predisposing polymorphism causes a sleep disorder characterized by decreased wakefulness.

7. The method of claim 5, wherein the predisposing polymorphism causes a sleep disorder characterized by increased wakefulness or insomnia.

8. The method of claim 5, wherein the disorder is narcolepsy.

9. The method of claim 1, wherein the disorder is selected from the group consisting of a mood disorder, chronic fatigue syndrome and an attention deficit disorder.

10. The method of claim 1, wherein the subject is human.

11. The method of claim 1, wherein the subject is canine.

12. The method of claim 11, wherein the polymorphism to be detected is within a genomic region between markers 26-8 and 530-3, inclusive, of canine chromosome 12.

13. A method of screening for biologically active agents that modulate sleep or wakefulness through modulation of hypocretin receptor activity, the method comprising:
combining a candidate agent with an isolated cell comprising a nucleic acid encoding a mammalian hypocretin receptor polypeptide;
determining the effect of said agent on hypocretin receptor activity;
wherein an agent that modulates hypocretin receptor activity and thus modulates sleep or wakefulness is identified where the agent increases or decreases hypocretin receptor activity.

14. The method of claim 13, wherein the candidate agent is a hypocretin receptor agonist and hypocretin receptor activity is detected by binding of the candidate agent to the hypocretin receptor.

15. The method of claim 13, wherein the agent is a hypocretin receptor antagonist and hypocretin receptor activity is detected by.

16. A method of screening for biologically active agents that modulate sleep or wakefulness through modulation of hypocretin receptor activity, the method comprising:
administering a candidate agent to a non-human animal model for function of an hypocretin receptor gene, the animal comprising a genetic alteration of a hypocretin receptor gene sequence or a hypocretin polypeptide sequence;
determining the effect of said agent on hypocretin receptor activity;
wherein an agent that modulates hypocretin receptor activity and thus modulates sleep or wakefulness is identified where the agent increases or decreases hypocretin receptor activity.

17. The method of claim 16, wherein said determining is by detecting an alteration in sleep pattern in the animal.

18. A method of treating a sleep disorder in a subject, the sleep disorder being characterized by decreased wakefulness relative to an unaffected subject, the method comprising:
administering to a subject having a sleep disorder associated with decreased wakefulness an amount of a hypocretin receptor agonist effective to increase wakefulness in the subject.

19. The method of claim 18, wherein the hypocretin receptor agonist is hypocretin or a hypocretin derivative.

20. The method of claim 18, wherein the sleep disorder is narcolepsy.

21. A method of treating a sleep disorder in a subject, the sleep disorder being characterized by increased wakefulness relative to an unaffected subject, the method comprising:
administering to a subject having a sleep disorder associated with increased wakefulness an amount of a hypocretin receptor antagonist effective to increase sleep in the subject.

22. A method of treating a subject having a hypocretin system disorder that causes at least one of depression, chronic fatigue syndrome or attention hyperactivity disorder, the method comprising:
administering to the subject an amount of a hypocretin receptor agonist sufficient to alleviate symptoms of the hypocretin system disorder.

23. A method for predicting the responsivity of a subject to administration of an agonist or antagonist of hypocretin receptor, wherein the subject suffers from a disorder selected from the group consisting of a sleep disorder, a mood disorder, chronic fatigue syndrome or an attention deficit disorder, the method comprising:
analyzing the genomic DNA or mRNA of a subject for the presence of at least one polymorphism selected from the group consisting of: a hypocretin receptor polymorphism and a hypocretin peptide polymorphism;
wherein the presence of the polymorphism indicates an increased probability that the subject suffers from a disorder that can be treated by administration of a hypocretin receptor agonist or hypocretin receptor antagonist.

24. A pharmaceutical composition comprising a hypocretin receptor agonist in an amount effective to promote wakefulness.

25. The pharmaceutical composition of claim 24, wherein the hypocretin receptor agonist is hypocretin or a hypocretin derivative.

26. A pharmaceutical composition comprising a hypocretin receptor antagonist in an amount effective to promote sleep.

27. A method for detecting a predisposition to a sleep disorder in an individual, the method comprising:
detecting an autoimmune response in a biological sample from a subject suspected of having or being susceptible to a sleep disorder, wherein the autoimmune response causes a decrease in binding of endogenous hypocretin to a hypocretin receptor or leads to destruction of hypocretin producing cells;
wherein detection of the autoimmune response is indicative of a sleep disorder in the subject.

28. The method of claim 27, wherein the autoimmune response is detected by detecting the presence of an auto antibody that specifically binds a hypocretin receptor.

29. The method of claim 27, wherein the autoimmune response is a cellular immune response is directed against a hypocretin receptor.

30. The method of claim 27, wherein the autoimmune response is directed against a component of a hypocretin-containing cell.

31. The method of claim 27, wherein the sleep disorder is narcolepsy.

32. A method for detecting a sleep disorder or a predisposition to a sleep disorder in an subject, the method comprising:
detecting a level of hypocretin in a biological sample from a test subject suspected of having or being susceptible to a sleep disorder;
wherein detection of a level of hypocretin in the sample that is altered relative to a level of hypocretin in a normal subject is indicative of a sleep disorder in the test subject.

33. The method of claim 32, wherein said detecting is by detection of binding of hypocretin-binding molecule to hypocretin in the test sample.

34. The method of claim 32, wherein said detecting is by detection of a biological activity of a peptide derived from the preprohypocretin gene.

35. The method of claim 32, wherein said detecting is by detection of an amount of hypocretin peptide in the sample.

36. The method of claim 32, wherein the sleep disorder is narcolepsy.

37. A method for detecting a hypocretin-related disorder or susceptibility to a hypocretin-related disorder in a subject, the hypocretin-related disorder being selected from the group consisting of a mood disorder, chronic fatigue syndrome, and attention deficit disorder, the method comprising:
detecting at least one of: a) a level of hypocretin peptide in a sample from a test subject, b) a level of expression of a hypocretin receptor in a sample obtained from a test subject, or c) a number of hypocretin-containing cells in tissue of a test subject, wherein the test subject is suspected of suffering from a hypocretin-related disorder;
wherein detection of a level of hypocretin peptide, a level of hypocretin receptor expression, or a number of hypocretin-containing cells that is altered relative to that found in a normal subject is indicative of a hypocretin-related disorder in the test subject.

38. An isolated nucleic acid molecule comprising at least 15 contiguous nucleotides and capable of hybridizing under high stringency conditions to a sequence encoding a mutated canine hypocretin receptor or a complement of said sequence encoding a mutated canine hypocretin receptor, which mutated hypocretin receptor causes canine narcolepsy.

39. The isolated nucleic acid molecule of claim 38, wherein the probe hybridizes specifically to a sequence encoding an amino acid having a sequence of SEQ ID NO:10.

40. The isolated nucleic acid molecule of claim 38, wherein the probe hybridizes specifically to a sequence encoding an amino acid having a sequence of SEQ ID NO:11.

41. The isolated nucleic acid molecule of claim 38 further characterized by specific hybridization to SEQ ID NO:13.

42. The isolated molecule of claim 38 further characterized by specific hybridization to SEQ ID NO:15.

43. A kit comprising the isolated nucleic acid molecule of claim 38, wherein the kit is useful in detecting a narcolepsy susceptibility locus in a canine subject.

44. A kit for use in detection of a canine narcolepsy susceptibility locus, the kit comprising at least one primer for amplification of a narcolepsy informative region, wherein the primer is selected from the group consisting of SEQ ID NOS:32-53.
